(19) United States
(12) Patent Application Publication
(10) Pub. No.: US 2024/0366101 A1
(43) Pub. Date: Nov. 7, 2024

(54) CARDIOVASCULAR AGE ESTIMATION

(71) Applicant: Whoop, Inc., Boston, MA (US)

(72) Inventors: Behnoosh Tavakoli, Needham, MA (US); Mostafa Ghannad-Rezaie, Malden, MA (US); Victoria Harrison Lee, Waltham, MA (US); Daphne Liu, Sandy, UT (US); Emily Rachel Capodilupo, Boston, MA (US); John Vincenzo Capodilupo, Boston, MA (US)

(21) Appl. No.: 18/772,785

(22) Filed: Jul. 15, 2024

Related U.S. Application Data
(63) Continuation of application No. 17/388,711, filed on Jul. 29, 2021.
(60) Provisional application No. 63/058,155, filed on Jul. 29, 2020.

(51) Int. Cl. A61B 5/024; A61B 5/00

(52) U.S. Cl. CPC A61B 5/02416; A61B 5/0022; A61B 5/02438; A61B 5/681; A61B 5/7264; A61B 2560/02; A61B 2560/0475

(57) ABSTRACT

The features of physiological signals can be mapped to a latent space in order to draw inferences about activity, health, and age of an individual. For example, heart rate pulses for a population can be acquired as PPG signals and features of these acquired PPG signals can be mapped to a latent space, along with biological age, in order to encode data in the latent space such that location and/or distance within the latent space can be used to infer a corresponding cardiovascular age. Features of a current pulse of PPG data for a user can then be transformed into the latent space with an autoencoder or the like in order to estimate a cardiovascular age for the user, which can also be compared to the user's biological age in order to draw inferences about fitness and/or provide recommendations, coaching, and the like.

### CROSS-REFERENCE TO RELATED APPLICATIONS

This application is a continuation of U.S. patent application Ser. No. 17/388,711 filed on Jul. 29, 2021, which claims priority to U.S. Provisional Application No. 63/058,155 filed on Jul. 29, 2020, where the entire content of each of the foregoing is hereby incorporated by reference.

### BACKGROUND

Photoplethysmography (PPG) is used in wearable technology to infer heart rate based on measurements of light absorption in the surface of the skin, which is correlated with blood volume in the skin. As the blood volume changes during a cardiac cycle, the corresponding PPG signal also changes in a manner that permits derivation of the heart rate. However, the pulse shape of a PPG signal contains a wealth of additional information. For example, the PPG pulse shape captured during systolic cycles may contain information about how quickly the heart muscle can contract, while the PPG pulse shape captured during diastolic cycles may have information about the compliance of vasculature and the function of the artery valves. There remains a need for improved techniques for deriving health and fitness information from the shape of PPG pulses and other physiological signals.

### SUMMARY

Variations in pulse shape over time can be used to draw inferences about activity, health, and age of an individual. For example, PPG pulses may be mapped to a latent space where variations in shape can be measured directly in terms of distance between pulses. In one aspect, pulse-to-pulse comparisons for an individual can be used to estimate strain, recovery, sleep, and so forth. Longer term measurements (e.g., over weeks, month, or years) can be used to detect changes in health and fitness for the individual. In another aspect, pulse-to-pulse comparisons among different individuals can be used to estimate relative cardiovascular health, age, and the like.

In an aspect, a computer program product disclosed herein may include computer executable code embodied in a non-transitory computer readable medium that, when executing on one or more computing devices, performs the steps of: acquiring pulse data including a plurality of heart pulse samples; creating a latent space using the pulse data based on one or more features of a characteristic pulse shape in the pulse data; receiving a first pulse of heart rate data and a second pulse of heart rate data from a wearable physiological monitor worn by a user during a window corresponding to the characteristic pulse shape; measuring a distance within the latent space between the first pulse and the second pulse; and calculating a fitness score for the user based on the distance within the latent space.

Implementations may include one or more of the following features. The wearable physiological monitor may acquire pulse data using photoplethysmography. The plurality of heart pulse samples may include heart pulse samples from the user. The plurality of heart pulse samples may include heart pulse samples from a large population of users. The characteristic pulse shape may include an average pulse shape for a population of users.

In an aspect, a method disclosed herein may include: capturing physiological data having a characteristic pulse shape; creating a latent space for one or more features of the characteristic pulse shape; receiving a first pulse of physiological data and a second pulse of physiological data during a window corresponding to the characteristic pulse shape; measuring a distance within the latent space between the first pulse and the second pulse; and calculating a fitness score for a person based on the distance within the latent space.

Implementations may include one or more of the following features. The method may further include receiving the first pulse of physiological data from a physiological monitor for the person. The first pulse of physiological data may include photoplethysmography data. The first pulse of physiological data may include heart rate data. The physiological monitor may include a wearable physiological monitor. The method may further include receiving the second pulse of physiological data from the physiological monitor for the person. The second pulse of physiological data may include an average pulse shape for a population of users. The second pulse of physiological data may include an average pulse shape for a history of an individual user. The fitness score may measure one or more of sleep, strain, and recovery. The fitness score may measure fitness for an individual. The fitness score may measure at least one of cardiovascular fitness and cardiovascular age relative to a population of users. The method may further include acquiring a data set of physiological pulses from a population of users. The method may further include normalizing the data set to a maximum pulse value. The method may further include applying the data set to train an autoencoder network on the latent space.

In an aspect, a system disclosed herein may include: a memory storing a latent space for an autoencoder that encodes a number of features of a photoplethysmography pulse signal based on a characteristic pulse shape of photoplethysmography pulse samples of a population, where the one or more features include a fitness level associated with a pulse sample; a wearable physiological monitor configured to acquire a first pulse sample of heart rate data and a second pulse sample of heart rate data from a user during a window corresponding to the characteristic pulse shape; and a processor configured to receive the first pulse sample and the second pulse sample, to encode the first pulse sample and the second pulse sample with the autoencoder into the latent space, to measure a distance within the latent space between the first pulse and the second pulse, and to calculate the fitness score for the user based on the distance within the latent space.

Implementations may include one or more of the following features. The processor and the memory may be in the wearable physiological monitor. The processor and the memory may reside on a remote resource configured to receive data through a data network from the wearable physiological monitor.

In an aspect, a computer program product disclosed herein may include computer executable code embodied in a non-transitory computer readable medium that, when executing on one or more computing devices, performs the steps of: acquiring pulse data including a plurality of heart pulse samples; training an autoencoder to encode the plurality of heart pulse samples into a latent space that differentiates among the plurality of heart samples according to an objective measure; storing a representation of the autoencoder and the latent space as a machine learning model in a memory of a wearable physiological monitoring device; acquiring a pulse sample from a user with the wearable physiological monitor; estimating objective measure information for the user with the machine learning model based on the pulse sample; and displaying the objective measure information to the user.

Implementations may include one or more of the following features. The objective measure may be one of an age and blood pressure level of the user. The objective measure may be a cardiovascular age of the user. The wearable physiological monitor may include a photoplethysmography system configured to acquire the pulse sample.

In an aspect, a method disclosed herein may include: acquiring pulse data including a plurality of heart pulse samples; training an autoencoder to encode the plurality of heart pulse samples into a latent space that differentiates among the plurality of heart samples according to an objective measure; storing a representation of an autoencoder and a latent space as a machine learning model configured to differentiate heart pulse shapes according to the objective measure; receiving a pulse sample from a user; and estimating an objective measure of the user with the machine learning model.

Implementations may include one or more of the following features. The method may further include displaying the objective measure to the user. The method may further include transmitting the objective measure to a remote server. Receiving the pulse sample may include acquiring the pulse sample with a wearable physiological monitor. Receiving the pulse sample may include acquiring the pulse sample with a photoplethysmography system. The method may further include comparing the objective measure to a reported objective measure for the user. The objective measure may be an age of the user. The method may further include evaluating a fitness of the user based on a difference between the age estimated by the machine learning model and a reported age provided by the user. The plurality of heart pulse samples may include data from a large population of at least one thousand subjects. The method may further include reporting a hardware error for a device acquiring the pulse sample when a representation of the pulse sample in the latent space lies outside a predetermined manifold for the objective measure. Training the autoencoder may include labelling a representation of each one of the plurality of heart pulse samples with a corresponding objective measure of a subject associated with the one of the plurality of heart pulse samples. The method may further include adjusting a fitness metric for the user based on the objective measure. The fitness metric may include a resting heart rate. The fitness metric may include a maximum heart rate. The fitness metric may include one or more of a sleep score, a recovery score, and a strain score.

In an aspect, a system disclosed herein may include: a memory storing an autoencoder and a latent space as a machine learning model configured to estimate an objective measure based on a pulse sample; a wearable physiological monitor configured to acquire the pulse sample from a user; and a processor configured to receive the pulse sample, to encode the pulse sample with the autoencoder, and to estimate an objective measure of the user based on a location of the pulse sample in the latent space.

Implementations may include one or more of the following features. The wearable physiological monitor may be configured to acquire the pulse sample with photoplethysmography. The processor and the memory may reside on the wearable physiological monitor.

### BRIEF DESCRIPTION OF THE DRAWINGS

The foregoing and other objects, features, and advantages of the devices, systems, and methods described herein will be apparent from the following description of particular embodiments thereof, as illustrated in the accompanying figures. The figures are not necessarily to scale, emphasis instead being placed upon illustrating the principles of the devices, systems, and methods described herein.

FIG. 1 illustrates front and back perspective views of a wearable system configured as a bracelet including one or more straps.

FIG. 2 shows a block diagram illustrating components of a wearable physiological measurement system configured to provide continuous collection and monitoring of physiological data.

FIG. 3 is a flow chart illustrating a signal processing algorithm for generating a sequence of heart rates for every detected heartbeat that may be embodied in computer-executable instructions stored on one or more non-transitory computer-readable media.

FIG. 4 is a flow chart illustrating a method of determining an intensity score.

FIG. 5 is a flow chart illustrating a method by which a user may use intensity and recovery scores.

FIG. 6 is a flow chart illustrating a method for detecting heart rate variability in sleep states.

FIG. 7 is a bottom view of a wearable, continuous physiological monitoring device.

FIG. 8 is a flow chart illustrating a method for estimating an objective measure with pulse data using a machine learning model.

FIG. 9 shows an exemplary autoencoder and decoder.

FIG. 10 illustrates an exemplary data set for training an autoencoder.

FIG. 11 illustrates exemplary latent spaces for pulse shape.

FIG. 12 is a flow chart illustrating a method for calculating a fitness level of a user using pulse data.

FIG. 13 illustrates a physiological monitoring system.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitations of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as including any deviation as would be appreciated by one of ordinary skill in the art to operate satisfactorily for an intended purpose, or where applicable, any acceptable range of deviation appropriate to a measurement of the numerical value or achievable by instrumentation used to measure the amount. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “above,” “below,” and the like, are words of convenience and are not to be construed as limiting terms.

Exemplary embodiments provide physiological measurement systems, devices and methods for continuous health and fitness monitoring, and provide improvements to overcome the drawbacks of conventional heart rate monitors. One aspect of the present disclosure is directed to providing a lightweight wearable system with a strap that collects various physiological data or signals from a wearer. The strap may be used to position the system on an appendage or extremity of a user, for example, wrist, ankle, and the like. Exemplary systems are wearable and enable real-time and continuous monitoring of heart rate without the need for a chest strap or other bulky equipment which could otherwise cause discomfort and prevent continuous wearing and use. The system may determine the user's heart rate without the use of electrocardiography and without the need for a chest strap. Exemplary systems can thereby be used in not only assessing general well-being but also in continuous monitoring of fitness. Exemplary systems also enable monitoring of one or more physiological parameters in addition to heart rate including, but not limited to, body temperature, heart rate variability, motion, sleep, stress, fitness level, recovery level, effect of a workout routine on health and fitness, caloric expenditure, and the like.

A health or fitness monitor that includes bulky components may hinder continuous wear. Existing fitness monitors often include the functionality of a watch, thereby making the health or fitness monitor quite bulky and inconvenient for continuous wear. Accordingly, one aspect is directed to providing a wearable health or fitness system that does not include bulky components, thereby making the bracelet slimmer, unobtrusive and appropriate for continuous wear. The ability to continuously wear the bracelet further allows continuous collection of physiological data, as well as continuous and more reliable health or fitness monitoring. For example, embodiments of the bracelet disclosed herein allow users to monitor data at all times, not just during a fitness session. In some embodiments, the wearable system may or may not include a display screen for displaying heart rate and other information. In other embodiments, the wearable system may include one or more light emitting diodes (LEDs) to provide feedback to a user and display heart rate selectively. In some embodiments, the wearable system may include a removable or releasable modular head that may provide additional features and may display additional information. Such a modular head can be releasably installed on the wearable system when additional information display is desired, and removed to improve the comfort and appearance of the wearable system. In other embodiments, the head may be integrally formed in the wearable system.

Exemplary embodiments also include computer-executable instructions that, when executed, enable automatic interpretation of one or more physiological parameters to assess the cardiovascular intensity experienced by a user (embodied in an intensity score or indicator) and the user's recovery after physical exertion or daily stress given sleep and other forms of rest (embodied in a recovery score). These indicators or scores may be stored and displayed in a meaningful format to assist a user in managing his health and exercise regimen.

In an exemplary technique of data transmission, data collected by a wearable system may be transmitted directly to a cloud-based data storage, from which data may be downloaded for display and analysis on a website. In another exemplary technique of data transmission, data collected by a wearable system may be transmitted via a mobile communication device application to a cloud-based data storage, from which data may be downloaded for display and analysis on a website.

The term “user” as used herein, refers to any type of animal, human or non-human, whose physiological information may be monitored using an exemplary wearable physiological monitoring system. The term “body,” as used herein, refers to the body of a user.

The term “continuous,” as used herein in connection with heart rate data collection, refers to collection of heart rate data at a sufficient frequency to enable detection of every heart beat and also refers to collection of heart rate data continuously throughout the day and night.

The term “computer-readable medium,” as used herein, refers to a non-transitory storage hardware, non-transitory storage device or non-transitory computer system memory that may be accessed by a controller, a microcontroller, a computational system or a module of a computational system to encode thereon computer-executable instructions or software programs. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

Exemplary embodiments provide wearable physiological measurements systems that are configured to provide continuous measurement of heart rate. Exemplary systems are configured to be continuously wearable on an appendage, for example, wrist or ankle, and do not rely on electrocardiography or chest straps in detection of heart rate. The exemplary system includes one or more light emitters for emitting light at one or more desired frequencies toward the user's skin, and one or more light detectors for received light reflected from the user's skin. The light detectors may include a photo-resistor, a photo-transistor, a photo-diode, and the like. As light from the light emitters (for example, green light) pierces through the skin of the user, the blood's natural absorbance or transmittance for the light provides fluctuations in the photo-resistor readouts. These waves have the same frequency as the user's pulse since increased absorbance or transmittance occurs only when the blood flow has increased after a heartbeat. The system includes a processing module implemented in software, hardware or a combination thereof for processing the optical data received at the light detectors and continuously determining the heart rate based on the optical data. The optical data may be combined with data from one or more motion sensors, e.g., accelerometers and/or gyroscopes, to minimize or eliminate noise in the heart rate signal caused by motion or other artifacts (or with other optical data of another wavelength).

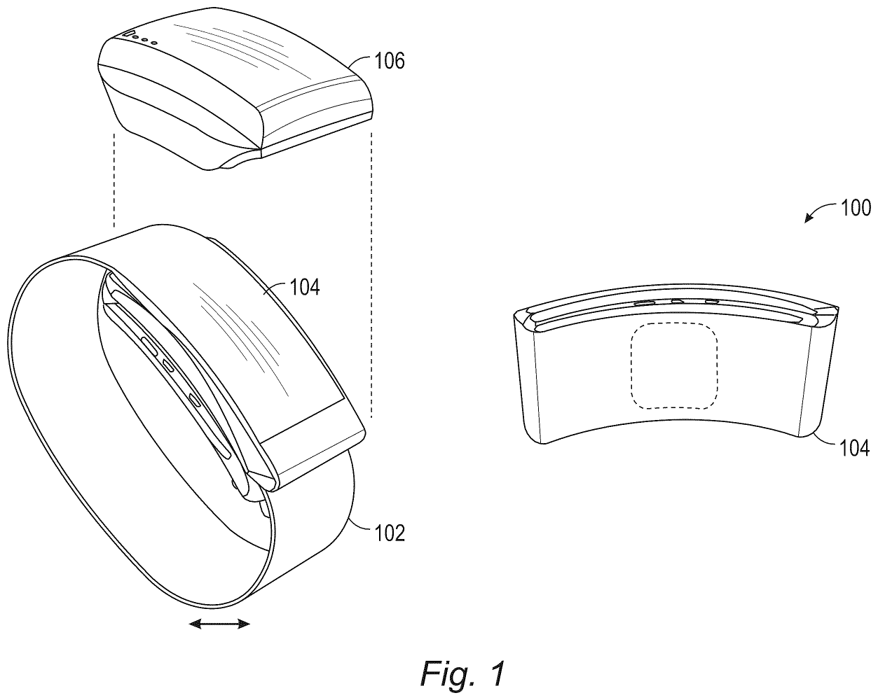

FIG. 1 illustrates front and back perspective views of one embodiment of a wearable system configured as a bracelet 100 including one or more straps 102. The bracelet 100 may be sleek and lightweight, thereby making it appropriate for continuous wear. The bracelet 100 may or may not include a display screen, e.g., user interface 106 such as a light emitting diode (LED) display for displaying any desired data (e.g., instantaneous heart rate).

As shown in the non-limiting embodiment in FIG. 1, the strap 102 of the bracelet 100 may have a wider side and a narrower side. In one embodiment, a user may simply insert the narrower side into the thicker side and squeeze the two together until the strap 102 is tight around the wrist. To remove the strap 102, a user may push the strap 102 further inwards, which unlocks the strap 102 and allows it to be released from the wrist. In other embodiments, various other fastening means may be provided. For example, the fastening mechanism may include, without limitation, a clasp, clamp, clip, dock, friction fit, hook and loop, latch, lock, pin, screw, slider, snap, button, spring, yoke, and so on.

In some embodiments, the strap 102 of the bracelet 100 may be a slim elastic band formed of any suitable elastic material, for example, rubber. Certain embodiments of the wearable system may be configured to have one size that fits all. Other embodiments may provide the ability to adjust for different wrist sizes. In one aspect, a combination of constant module strap material, a spring-loaded, floating optical system and a silicon-rubber finish may be used in order to achieve coupling while maintaining the strap's comfort for continuous use. Use of medical-grade materials to avoid skin irritations may be utilized.

As shown in FIG. 1, the wearable system (e.g., the bracelet 100) may include components configured to provide various functions such as data collection and streaming functions of the system. In some embodiments, the wearable system may include a button underneath the wearable system. In some embodiments, the button may be configured such that, when the wearable system is properly tightened to one's wrist, the button may press down and activate the system to begin storing information. In other embodiments, the button may be disposed and configured such that it may be pressed manually at the discretion of a user to begin storing information or otherwise to mark the start or end of an activity period. In some embodiments, the button may be held to initiate a time stamp and held again to end a time stamp, which may be transmitted, directly or through a mobile communication device application, to a website as a time stamp.

Time stamp information may be used, for example, as a privacy setting to indicate periods of activity during which physiological data may not be shared with other users. In one aspect, the button may be tapped, double-tapped (or triple-tapped or more), or held down in order to perform different functions or display different information (e.g., display battery information, generate time stamps, etc.). Other implementations may include more or less buttons or other forms of interfaces. More general, a privacy switch such as any of the user inputs or controls described herein may be operated to control restrictions on sharing, distribution, or use of heart rate or other continuously monitored physiological data. For example, the privacy switch may include a toggle switch to switch between a private setting where data is either not gathered at all or where data is stored locally for a user, and between a public, shared, or other non-private setting where data is communicated over a network and/or to a shared data repository. The privacy switch may also support numerous levels of privacy, e.g., using a hierarchical, role-based, and/or identity-based arrangement of permitted users and/or uses. As another example, various levels of privacy may be available for the type and amount of data that is shared versus private. In general, the privacy switch may be a physical switch on the wearable system, or a logical switch or the like maintained on a computer or other local or mobile computing device of the user, or on a website or other network-accessible resource where the user can select and otherwise control privacy settings for monitored physiological data.

In some embodiments, the wearable system may be waterproof so that users never need to remove it, thereby allowing for continuous wear.

The wearable system may include a heart rate monitor. In one example, the heart rate may be detected from the radial artery. See, Certified Nursing Association, “Regular monitoring of your patient's radial pulse can help you detect changes in their condition and assist in providing potentially life-saving care.” See, http://cnatraininghelp.com/cna-skills/counting-and-recording-a-radial-pulse, the entire contents of which are incorporated herein by reference. Thus, the wearable system may include a pulse sensor. In one embodiment, the wearable system may be configured such that, when a user wears it around their wrist and tightens it, the sensor portion of the wearable system is secured over the user's radial artery or other blood vessel. Secure connection and placement of the pulse sensor over the radial artery or other blood vessel may allow measurement of heart rate and pulse. It will be understood that this configuration is provided by way of example only, and that other sensors, sensor positions, and monitoring techniques may also or instead be employed without departing from the scope of this disclosure.

In some embodiments, the pulse or heart rate may be taken using an optical sensor coupled with one or more light emitting diodes (LEDs), all directly in contact with the user's wrist. The LEDs are provided in a suitable position from which light can be emitted into the user's skin. In one example, the LEDs mounted on a side or top surface of a circuit board in the system to prevent heat buildup on the LEDs and to prevent burns on the skin. The circuit board may be designed with the intent of dissipating heat, e.g., by including thick conductive layers, exposed copper, heatsink, or similar. In one aspect, the pulse repetition frequency is such that the amount of power thermally dissipated by the LED is negligible. Cleverly designed clastic wrist straps can ensure that the sensors are always in contact with the skin and that there is a minimal amount of outside light seeping into the sensors. In addition to the elastic wrist strap, the design of the strap may allow for continuous micro adjustments (no preset sizes) in order to achieve an optimal fit, and a floating sensor module. The sensor module may be free to move with the natural movements caused by flexion and extension of the wrist.

In some embodiments, the wearable system may be configured to record other physiological parameters including, but not limited to, skin temperature (using a thermometer), galvanic skin response (using a galvanic skin response sensor), motion (using one or more multi-axes accelerometers and/or gyroscope), and the like, and environmental or contextual parameters, e.g., ambient temperature, humidity, time of day, and the like. In an implementation, sensors are used to provide at least one of continuous motion detection, environmental temperature sensing, electrodermal activity (EDA) sensing, galvanic skin response (GSR) sensing, and the like. In this manner, an implementation can identify the cause of a detected physiological event. Reflectance PhotoPlethysmoGraphy (RPPG) may be used for the detection of cardiac activity, which may provide for non-intrusive data collection, usability in wet, dusty and otherwise harsh environments, and low power requirements. For example, as explained herein, using the physiological readouts of the device and the analytics described herein, an “Intensity Score” (e.g., 0-21) (e.g., that measures a user's recent exertion), a “Recovery Score” (e.g., 0-100%), and “Sleep Score” (e.g., 0-100) may together measure readiness for physical and psychological exertion.

In some embodiments, the wearable system may further be configured such that a button underneath the system may be pressed against the user's wrist, thus triggering the system to begin one or more of collecting data, calculating metrics and communicating the information to a network. In some embodiments, the sensor used for, e.g., measuring heart rate or GSR or any combination of these, may be used to indicate whether the user is wearing the wearable system or not. In some embodiments, power to the one or more LEDs may be cut off as soon as this situation is detected and reset once the user has put the wearable system back on their wrist.

The wearable system may include one, two, or more sources of battery life, e.g., two or more batteries. In some embodiments, it may have a battery that can slip in and out of the head of the wearable system and can be recharged using an included accessory. Additionally, the wearable system may have a built-in battery that is less powerful. When the more powerful battery is being charged, the user does not need to remove the wearable system and can still record data (during sleep, for example).

In some embodiments, an application associated with data from an exemplary wearable system (e.g., a mobile communication device application) may include a user input component for enabling additional contextual data, e.g., emotional (e.g., the user's feelings), perceived intensity, and the like. When the data is uploaded from the wearable system directly or indirectly to a website, the website may record a user's “Vibes” alongside their duration of exercise and sleep.

In exemplary embodiments, the wearable system is enabled to automatically detect when the user is asleep, awake but at rest and exercising based on physiological data collected by the system.

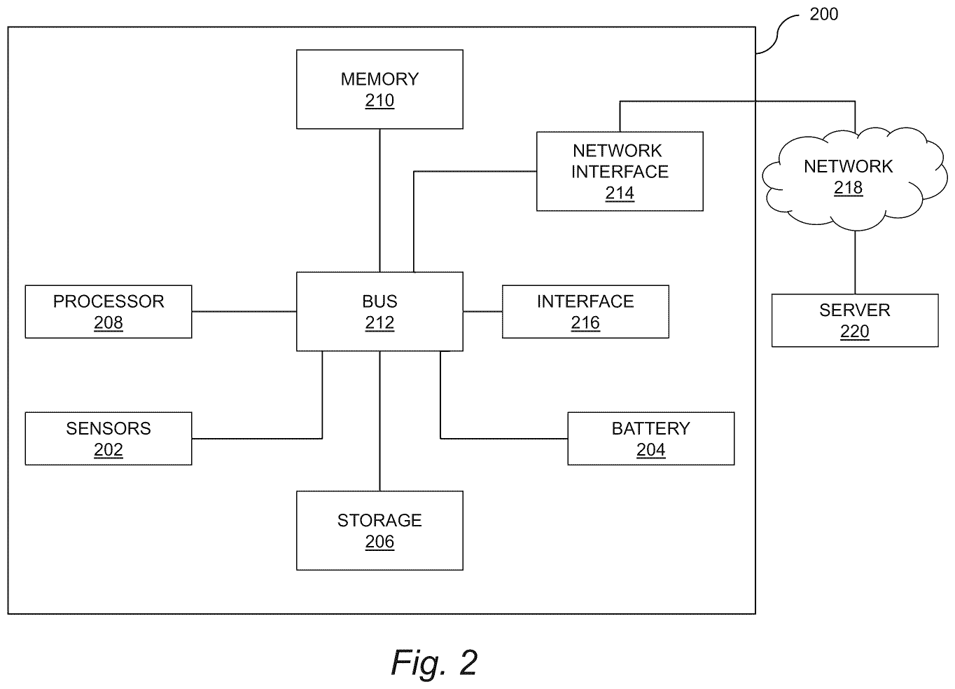

FIG. 2 shows a block diagram illustrating exemplary components of a wearable physiological measurement system 200 configured to provide continuous collection and monitoring of physiological data. The wearable system 200 includes one or more sensors 202. As discussed above, the sensors 202 may include a heart rate monitor. In some embodiments, the wearable system 200 may further include one or more of sensors for detecting calorie burn, distance and activity. Calorie burn may be based on a user's heart rate, and a calorie burn measurement may be improved if a user chooses to provide his or her weight and/or other physical parameters. In some embodiments, manual entering of data is not required in order to derive calorie burn; however, data entry may be used to improve the accuracy of the results. In some embodiments, if a user has forgotten to enter a new weight, he/she can enter it for past weeks and the calorie burn may be updated accordingly.

The sensors 202 may include one or more sensors for activity measurement. In some embodiments, the system may include one or more multi-axes accelerometers and/or gyroscope to provide a measurement of activity. In some embodiments, the accelerometer may further be used to filter a signal from the optical sensor for measuring heart rate and to provide a more accurate measurement of the heart rate. In some embodiments, the wearable system may include a multi-axis accelerometer to measure motion and calculate distance, whether it be in real terms as steps or miles or as a converted number. Activity sensors may be used, for example, to classify or categorize activity, such as walking, running, performing another sport, standing, sitting or lying down. In some embodiments, one or more of collected physiological data may be aggregated to generate an aggregate activity level. For example, heart rate, calorie burn, and distance may be used to derive an aggregate activity level. The aggregate level may be compared with or evaluated relative to previous recordings of the user's aggregate activity level, as well as the aggregate activity levels of other users.

The sensors 202 may include a thermometer for monitoring the user's body or skin temperature. In one embodiment, the sensors may be used to recognize sleep based on a temperature drop, GSR data, lack of activity according to data collected by the accelerometer, and reduced heart rate as measured by the heart rate monitor. The body temperature, in conjunction with heart rate monitoring and motion, may be used to interpret whether a user is sleeping or just resting, as body temperature drops significantly when an individual is about to fall asleep), and how well an individual is sleeping as motion indicates a lower quality of sleep. The body temperature may also be used to determine whether the user is exercising and to categorize and/or analyze activities.

The system 200 includes one or more batteries 204. According to one embodiment, the one or more batteries may be configured to allow continuous wear and usage of the wearable system. In one embodiment, the wearable system may include two or more batteries. The system may include a removable battery that may be recharged using a charger. In one example, the removable battery may be configured to slip in and out of a head portion of the system, attach onto the bracelet, or the like. In one example, the removable battery may be able to power the system for around a week. Additionally, the system may include a built-in battery. The built-in battery may be recharged by the removable battery. The built-in battery may be configured to power the bracelet for around a day on its own. When the more removable battery is being charged, the user does not need to remove the system and may continue collecting data using the built-in battery. In other embodiments, the two batteries may both be removable and rechargeable.

In some embodiments, the system 200 may include a battery that is a wireless rechargeable battery. For example, the battery may be recharged by placing the system or the battery on a rechargeable mat. In other example, the battery may be a long range wireless rechargeable battery. In other embodiments, the battery may be a rechargeable via motion. In yet other embodiments, the battery may be rechargeable using a solar energy source.

The wearable system 200 includes one or more non-transitory computer-readable media 206 for storing raw data detected by the sensors of the system and processed data calculated by a processing module of the system.

The system 200 includes a processor 208, a memory 210, a bus 212, a network interface 214, and an interface 216. The network interface 214 is configured to wirelessly communicate data to an external network 218. The network 218 may include any communication network through which computer systems may exchange data. For example, the network 218 may include, but is not limited to, the Internet, an intranet, a LAN (Local Area Network), a WAN (Wide Area Network), a MAN (Metropolitan Area Network), a wireless network, an optical network, and the like. To exchange data via the network 218, the system 200 and the network 218 may use various methods, protocols and standards including, but not limited to, token ring, Ethernet, wireless Ethernet, Bluetooth, TCP/IP, UDP, HTTP, FTP, SNMP, SMS, MMS, SS7, JSON, XML, REST, SOAP, CORBA, IIOP, RMI, DCOM and Web Services. To ensure data transfer is secure, the system 200 may transmit data via the network using a variety of security measures including, but not limited to, TSL, SSL and VPN.

Some embodiments of the wearable system may be configured to stream information wirelessly to a social network. In some embodiments, data streamed from a user's wearable system to an external network 218 may be accessed by the user via a website. The network interface may be configured such that data collected by the system may be streamed wirelessly. In some embodiments, data may be transmitted automatically, without the need to manually press any buttons. In some embodiments, the system may include a cellular chip built into the system. In one example, the network interface may be configured to stream data using Bluetooth technology. In another example, the network interface may be configured to stream data using a cellular data service, such as via a 3G or 4G cellular network.

The system 200 may be coupled to one or more servers 220 via a communication network 218.

In some embodiments, a physiological measurement system may be configured in a modular design to enable continuous operation of the system in monitoring physiological information of a user wearing the system. The module design may include a strap and a separate modular head portion or housing that is removably couplable to the strap.

In the non-limiting illustrative module design, the strap 102 of a physiological measurement system may be provided with a set of components that enables continuous monitoring of at least a heart rate of the user so that it is independent and fully self-sufficient in continuously monitoring the heart rate without requiring the modular head portion 104. In one embodiment, the strap includes a plurality of light emitters for emitting light toward the user's skin, a plurality of light detectors for receiving light reflected from the user's skin, an electronic circuit board comprising a plurality of electronic components configured for analyzing data corresponding to the reflected light to automatically and continually determine a heart rate of the user, and a first set of one or more batteries for supplying electrical power to the light emitters, the light detectors and the electronic circuit board. In some embodiments, the strap may also detect one or more other physiological characteristics of the user including, but not limited to, temperature, galvanic skin response, and the like.

Certain exemplary systems may be configured to be coupled to any desired part of a user's body so that the system may be moved from one portion of the body (e.g., wrist) to another portion of the body (e.g., ankle) without affecting its function and operation. In one embodiment, the identity of the portion of the user's body to which the wearable system is attached may be determined based on one or more parameters including, but not limited to, absorbance level of light as returned from the user's skin, reflectance level of light as returned from the user's skin, motion sensor data (e.g., accelerometer and/or gyroscope), altitude of the wearable system, and the like.

In some embodiments, the processing module is configured to determine that the wearable system is taken off from the user's body. In one example, the processing module may determine that the wearable system has been taken off if data from the galvanic skin response sensor indicates data atypical of a user's skin. If the wearable system is determined to be taken off from the user's body, the processing module is configured to deactivate the light emitters and the light detectors and cease monitoring of the heart rate of the user to conserve power.

Exemplary systems include a processing module configured to filter the raw photoplethysmography data received from the light detectors to minimize contributions due to motion, and subsequently process the filtered data to detect peaks in the data that correspond with heart beats of a user. The overall algorithm for detecting heart beats takes as input the analog signals from optical sensors (mV) and accelerometer, and outputs an implied beats per minute (heart rate) of the signal accurate within a few beats per minute as that determined by an electrocardiography machine even during motion.

In one aspect, using multiple LEDs with different wavelengths reacting to movement in different ways can allow for signal recovery with standard signal processing techniques. The availability of accelerometer information can also be used to compensate for coarse movement signal corruption. In order to increase the range of movements that the algorithm can successfully filter out, an aspect utilizes techniques that augment the algorithm already in place. For example, filtering violent movements of the arm during very short periods of time, such as boxing as exercising, may be utilized by the system. By selective sampling and interpolating over these impulses, an aspect can account for more extreme cases of motion. Additionally, an investigation into different LED wavelengths, intensities, and configurations can allow the systems described herein to extract a signal across a wide spectrum of skin types and wrist sizes. In other words, motion filtering algorithms and signal processing techniques may assist in mitigating the risk caused by movement.

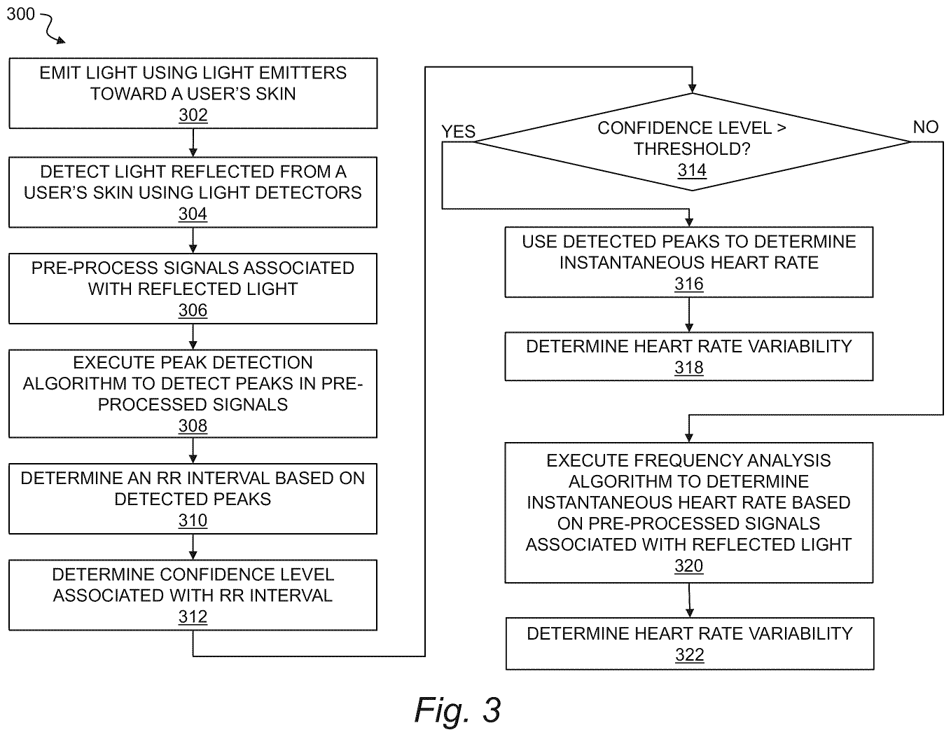

FIG. 3 is a flow chart illustrating a signal processing algorithm for generating a sequence of heart rates for every detected heartbeat that is embodied in computer-executable instructions stored on one or more non-transitory computer-readable media. As shown in step 302, the method 300 may include light emitters of a wearable physiological measurement system emitting light toward a user's skin. As shown in step 304, the method 300 may include detecting light reflected from the user's skin at the light detectors in the system. As shown in step 306, the method 300 may include pre-processing signals or data associated with the reflected light using any suitable technique to facilitate detection of heart beats. As shown in step 308, the method 300 may include a processing module of the system executing one or more computer-executable instructions associated with a peak detection algorithm to process data corresponding to the reflected light to detect a plurality of peaks associated with a plurality of beats of the user's heart. As shown in step 310, the method 300 may include the processing module determining an RR interval based on the plurality of peaks detected by the peak detection algorithm. As shown in step 312, the method 300 may include the processing module determining a confidence level associated with the RR interval.

Based on the confidence level associated with the RR interval estimate, the processing module selects either the peak detection algorithm or a frequency analysis algorithm to process data corresponding to the reflected light to determine the sequence of instantaneous heart rates of the user. The frequency analysis algorithm may process the data corresponding to the reflected light based on the motion of the user detected using, for example, an accelerometer. The processing module may select the peak detection algorithm or the frequency analysis algorithm regardless of a motion status of the user. It is advantageous to use the confidence in the estimate in deciding whether to switch to frequency-based methods as certain frequency-based approaches are unable to obtain accurate RR intervals for heart rate variability analysis. Therefore, an implementation maintains the ability to obtain the RR intervals for as long as possible, even in the case of motion, thereby maximizing the information that can be extracted.

For example, as shown in step 314, the method 300 may include determining whether the confidence level associated with the RR interval is above (or equal to or above) a threshold. In certain embodiments, the threshold may be predefined, for example, about 50%-90% in some embodiments and about 80% in one non-limiting embodiment. In other embodiments, the threshold may be adaptive, i.e., the threshold may be dynamically and automatically determined based on previous confidence levels. For example, if one or more previous confidence levels were high (i.e., above a certain level), the system may determine that a present confidence level that is relatively low compared to the previous levels is indicative of a less reliable signal. In this case, the threshold may be dynamically adjusted to be higher so that a frequency-based analysis method may be selected to process the less reliable signal.

If the confidence level is above (or equal to or above) the threshold, as shown in step 316, the method 300 may include the processing module using the plurality of peaks to determine an instantaneous heart rate of the user. On the other hand, as shown in step 320, the method 300 may include, based on a determination that the confidence level associated with the RR interval is equal to or below the predetermined threshold, the processing module executing one or more computer-executable instructions associated with the frequency analysis algorithm to determine an instantaneous heart rate of the user. The confidence threshold may be dynamically set based on previous confidence levels.

In some embodiments, as shown in step 318 and 322, the method 300 may include the processing module determining a heart rate variability of the user based on the sequence of the instantaneous heart rates/beats.

The system may include a display device configured to render a user interface for displaying the sequence of the instantaneous heart rates of the user, the RR intervals and/or the heart rate variability determined by the processing module. The system may include a storage device configured to store the sequence of the instantaneous heart rates, the RR intervals and/or the heart rate variability determined by the processing module.

In one aspect, the system may switch between different analytical techniques for determining a heart rate such as a statistical technique for detecting a heart rate and a frequency domain technique for detecting a heart rate. These two different modes have different advantages in terms of accuracy, processing efficiency, and information content, and as such may be useful at different times and under different conditions. Rather than selecting one such mode or technique as an attempted optimization, the system may usefully switch back and forth between these differing techniques, or other analytical techniques, using a predetermined criterion. An exemplary statistical technique employs probabilistic peak detection. An exemplary frequency analysis algorithm used in an implementation isolates the highest frequency components of the optical data, checks for harmonics common in both the accelerometer data and the optical data, and performs filtering of the optical data. This latter algorithm may, for example, take as input raw analog signals from the accelerometer (3-axis) and pulse sensors, and output heart rate values or beats per minute (BPM) for a given period of time related to the window of the spectrogram.

The exemplary wearable system computes heart rate variability (HRV) to obtain an understanding of the recovery status of the body. These values are captured right before a user awakes or when the user is not moving, in both cases photoplethysmography (PPG) variability yielding equivalence to the ECG HRV. HRV is traditionally measured using an ECG machine and obtaining a time series of R-R intervals. Because an exemplary wearable system utilizes photoplethysmography (PPG), it does not obtain the electric signature from the heart beats; instead, the peaks in the obtained signal correspond to arterial blood volume. At rest, these peaks are directly correlated with cardiac cycles, which enables the calculation of HRV via analyzing peak-to-peak intervals (the PPG analog of RR intervals). It has been demonstrated in medical literature that these peak-to-peak intervals, the “PPG variability,” is identical to ECG HRV while at rest.

An exemplary system may include a processing module that is configured to automatically adjust one or more operational characteristics of the light emitters and/or the light detectors to minimize power consumption while ensuring that all heart beats of the user are reliably and continuously detected. The operational characteristics may include, but are not limited to, a frequency of light emitted by the light emitters, the number of light emitters activated, a duty cycle of the light emitters, a brightness of the light emitters, a sampling rate of the light detectors, and the like. The processing module may adjust the operational characteristics based on one or more signals or indicators obtained or derived from one or more sensors in the system including, but not limited to, a motion status of the user, a sleep status of the user, historical information on the user's physiological and/or habits, an environmental or contextual condition (e.g., ambient light conditions), a physical characteristic of the user (e.g., the optical characteristics of the user's skin), and the like.

In one embodiment, the processing module may receive data on the motion of the user using, for example, an accelerometer. The processing module may process the motion data to determine a motion status of the user which indicates the level of motion of the user, for example, exercise, light motion (e.g., walking), no motion or rest, sleep, and the like. The processing module may adjust the duty cycle of one or more light emitters and the corresponding sampling rate of the one or more light detectors based on the motion status. For example, light emitters for PPG may be activated at a duty cycle ranging from about 1% to about 100%. In another example, the light emitters may be activated at a duty cycle ranging from about 20% to about 50% to minimize power consumption. Certain exemplary sampling rates of the light detectors may range from about 50 Hz to about 800 Hz, but are not limited to these exemplary rates. Certain non-limiting sampling rates are, for example, about 100 Hz, 200 Hz, 500 Hz, and the like.

In one non-limiting example, the light detectors may sample continuously when the user is performing an exercise routine so that the error standard deviation is kept within 5 beats per minute (BPM). When the user is at rest, the light detectors may be activated for about a 1% duty cycle—10 milliseconds each second (i.e., 1% of the time) so that the error standard deviation is kept within 5 BPM (including an error standard deviation in the heart rate measurement of 2 BPM and an error standard deviation in the heart rate changes between measurement of 3 BPM). When the user is in light motion (e.g., walking), the light detectors may be activated for about a 10% duty cycle—100 milliseconds each second (i.e., 10% of the time) so that the error standard deviation is kept within 6 BPM (including an error standard deviation in the heart rate measurement of 2 BPM and an error standard deviation in the heart rate changes between measurement of 4 BPM).

The processing module may adjust the brightness of one or more light emitters by adjusting the current supplied to the light emitters. For example, a first level of brightness may be set by current ranging between about 1 mA to about 10 mA, but is not limited to this exemplary range. A second higher level of brightness may be set by current ranging from about 11 mA to about 30 mA, but is not limited to this exemplary range. A third higher level of brightness may be set by current ranging from about 80 mA to about 120 mA, but is not limited to this exemplary range. In one non-limiting example, first, second and third levels of brightness may be set by current of about 5 mA, about 20 mA and about 100 mA, respectively.

Shorter-wavelength LEDs may require more power than is required by other types of heart rate sensors, such as, a piezo-sensor or an infrared sensor. Therefore, an exemplary wearable system may provide and use a unique combination of sensors-one or more light detectors for periods where motion is expected and one or more piezo and/or infrared sensors for low motion periods (e.g., sleep)—to save battery life. Certain other embodiments of a wearable system may exclude piezo-sensors and/or infrared sensors.

For example, upon determining that the motion status indicates that the user is at a first higher level of motion (e.g., exercising), one or more light emitters may be activated to emit light at a first wavelength. Upon determining that the motion status indicates that the user is at a second lower level of motion (e.g., at rest), non-light based sensors may be activated. The threshold levels of motion that trigger adjustment of the type of sensor may be based on one or more factors including, but are not limited to, skin properties, ambient light conditions, and the like.

The system may determine the type of sensor to use at a given time based on the level of motion (e.g., via an accelerometer) and whether the user is asleep (e.g., based on movement input, skin temperature and heart rate). Based on a combination of these factors the system selectively chooses which type of sensor to use in monitoring the heart rate of the user. Common symptoms of being asleep are periods of no movement or small bursts of movement (such as shifting in bed), lower skin temperature (although it is not a dramatic drop from normal), drastic GSR changes, and heart rate that is below the typical resting heart rate when the user is awake. These variables depend on the physiology of a person and thus a machine learning algorithm is trained with user-specific input to determine when he/she is awake/asleep and determine from that the exact parameters that cause the algorithm to deem someone asleep.

In an exemplary configuration, the light detectors may be positioned on the underside of the wearable system and all of the heart rate sensors may be positioned adjacent to each other. For example, the low power sensor(s) may be adjacent to the high power sensor(s) as the sensors may be chosen and placed where the strongest signal occurs. In one example configuration, a 3-axis accelerometer may be used that is located on the top part of the wearable system. In some embodiments, an operational characteristic of the microprocessor may be automatically adjusted to minimize power consumption. This adjustment may be based on a level of motion of the user's body.

More generally, the above description contemplates a variety of techniques for sensing conditions relating to heart rate monitoring or related physiological activity either directly (e.g., confidence levels or accuracy of calculated heart rate) or indirectly (e.g., motion detection, temperature). However measured, these sensed conditions can be used to intelligently select from among a number of different modes, including hardware modes, software modes, and combinations of the foregoing, for monitoring heart rate based on, e.g., accuracy, power usage, detected activity states, and so forth. Thus there is disclosed herein techniques for selecting from among two or more different heart rate monitoring modes according to a sensed condition.

Exemplary embodiments provide an analytics system for providing qualitative and quantitative monitoring of a user's body, health and physical training. The analytics system is implemented in computer-executable instructions encoded on one or more non-transitory computer-readable media. The analytics system relies on and uses continuous data on one or more physiological parameters including, but not limited to, heart rate. The continuous data used by the analytics system may be obtained or derived from an exemplary physiological measurement system disclosed herein, or may be obtained or derived from a derived source or system, for example, a database of physiological data. In some embodiments, the analytics system computes, stores and displays one or more indicators or scores relating to the user's body, health and physical training including, but not limited to, an intensity score and a recovery score. The scores may be updated in real-time and continuously or at specific time periods, for example, the recovery score may be determined every morning upon waking up, the intensity score may be determined in real-time or after a workout routine or for an entire day.

In certain exemplary embodiments, a fitness score may be automatically determined based on the physiological data of two or more users of exemplary wearable systems.

An intensity score or indicator provides an accurate indication of the cardiovascular intensities experienced by the user during a portion of a day, during the entire day or during any desired period of time (e.g., during a week or month). The intensity score is customized and adapted for the unique physiological properties of the user and takes into account, for example, the user's age, gender, anaerobic threshold, resting heart rate, maximum heart rate, and the like. If determined for an exercise routine, the intensity score provides an indication of the cardiovascular intensities experienced by the user continuously throughout the routine. If determined for a period of including and beyond an exercise routine, the intensity score provides an indication of the cardiovascular intensities experienced by the user during the routine and also the activities the user performed after the routine (e.g., resting on the couch, active day of shopping) that may affect their recovery or exercise readiness.

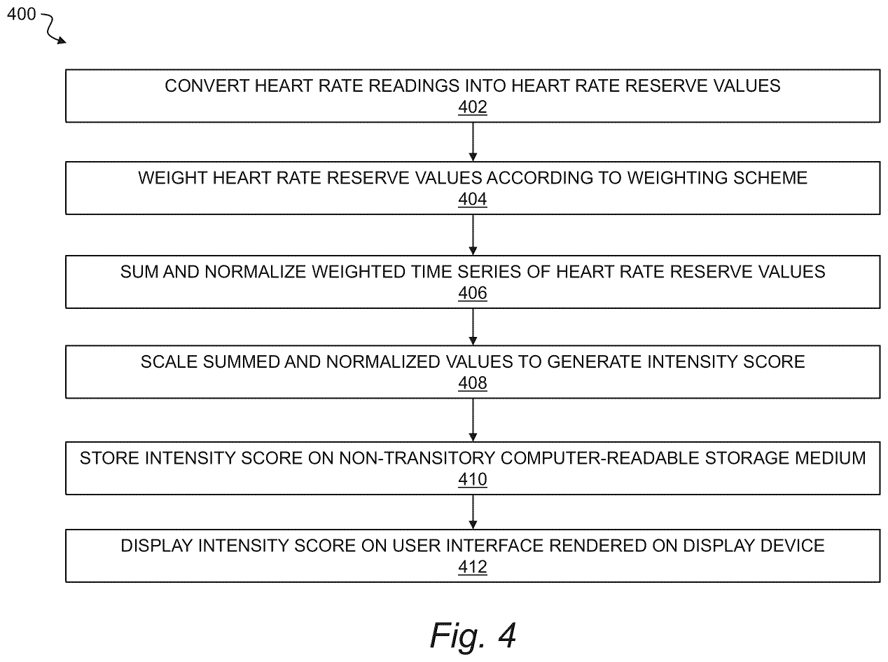

In exemplary embodiments, the intensity score is calculated based on the user's heart rate reserve (HRR) as detected continuously throughout the desired time period, for example, throughout the entire day. In one embodiment, the intensity score is an integral sum of the weighted HRR detected continuously throughout the desired time period. FIG. 4 is a flow chart illustrating a method of determining an intensity score.

As shown in step 402, the method 400 may include converting continuous heart rate readings to HRR values. A time series of heart rate data used in step 402 may be denoted as:

H∈T

A time series of HRR measurements, v(t), may be defined in the following expression in which MHR is the maximum heart rate and RHR is the resting heart rate of the user:

$$v ( t ) = \frac{H ( t ) - RHR}{NHR - RHR}$$

As shown in step 404, the method 400 may include weighting the HRR values according to a suitable weighting scheme. Cardiovascular intensity, indicated by an intensity score, is defined in the following expression in which w is a weighting function of the HRR measurements:

$$I ( {t}_{o} , {t}_{1} ) = {\int }_{{t}_{o}}^{{t}_{1}} w ( v ( t ) ) dt$$

As shown in step 406, the method 400 may include summing and normalizing the weighted time series of HRR values.

$${I}_{t} = {\int }_{T} w ( v ( t ) ) dt \leq w ( 1 ) \vert T \vert$$

Thus, the weighted sum is normalized to the unit interval, i.e., [0, 1]

$${N}_{T} = \frac{{I}_{T}}{w ( 1 ) \cdot 24 hr}$$

As shown in step 408, the method 400 may include scaling the summed and normalized values to generate user-friendly intensity score values. That is, the unit interval is transformed to have any desired distribution in a scale (e.g., a scale including 21 points from 0 to 21), for example, arctangent, sigmoid, sinusoidal, and the like. In certain distributions, the intensity values increase at a linear rate along the scale, and in others, at the highest ranges the intensity values increase at more than a linear rate to indicate that it is more difficult to climb in the scale toward the extreme end of the scale. In some embodiments, the raw intensity scores are scaled by fitting a curve to a selected group of “canonical” exercise routines that are predefined to have particular intensity scores.

In one embodiment, monotonic transformations of the unit interval are achieved to transform the raw HRR values to user-friendly intensity scores. An exemplary scaling scheme, expressed as ƒ: [0, 1]→[0, 1], is performed using the following function:

$$( x , N , p ) = 0.5 ( \frac{arctan ( N ( x - p ) )}{π / 2} + 1 )$$

To generate an intensity score, the resulting value may be multiplied by a number based on the desired scale of the intensity score. For example, if the intensity score is graduated from zero to 21, then the value may be multiplied by 21.

As shown in step 410, the method 400 may include storing the intensity score values on a non-transitory storage medium for retrieval, display and usage. As shown in step 412, the method 400 may include displaying the intensity score values on a user interface rendered on a visual display device. The intensity score values may be displayed as numbers and/or with the aid of graphical tools, e.g., a graphical display of the scale of intensity scores with current score, and the like. In some embodiments, the intensity score may be indicated by audio. In step 412, the intensity score values may be, in some embodiments, displayed along with one or more quantitative or qualitative pieces of information on the user including, but not limited to, whether the user has exceeded his/her anaerobic threshold, the heart rate zones experienced by the user during an exercise routine, how difficult an exercise routine was in the context of the user's training, the user's perceived exertion during an exercise routine, whether the exercise regimen of the user should be automatically adjusted (e.g., made easier if the intensity scores are consistently high), whether the user is likely to experience soreness the next day and the level of expected soreness, characteristics of the exercise routine (e.g., how difficult it was for the user, whether the exercise was in bursts or activity, whether the exercise was tapering, etc.), and the like. In one embodiment, the analytics system may automatically generate, store and display an exercise regimen customized based on the intensity scores of the user.

Step 406 may use any of a number of exemplary static or dynamic weighting schemes that enable the intensity score to be customized and adapted for the unique physiological properties of the user. In one exemplary static weighting scheme, the weights applied to the HRR values are based on static models of a physiological process. The human body employs different sources of energy with varying efficiencies and advantages at different HRR levels. For example, at the anaerobic threshold (AT), the body shifts to anaerobic respiration in which the cells produce two adenosine triphosphate (ATP) molecules per glucose molecule, as opposed to 36 at lower HRR levels. At even higher HRR levels, there is a further subsequent threshold (CPT) at which creatine triphosphate (CTP) is employed for respiration with even less efficiency.

In order to account for the differing levels of cardiovascular exertion and efficiency at the different HRR levels, in one embodiment, the possible values of HRR are divided into a plurality of categories, sections or levels (e.g., three) dependent on the efficiency of cellular respiration at the respective categories. The HRR parameter range may be divided in any suitable manner, such as, piecewise, including piecewise-linear, piecewise-exponential, and the like. An exemplary piecewise-linear division of the HRR parameter range enables weighting each category with strictly increasing values. This scheme captures an accurate indication of the cardiovascular intensity experienced by the user because it is more difficult to spend time at higher HRR values, which suggests that the weighting function should increase at the increasing weight categories.

In one non-limiting example, the HRR parameter range may be considered a range from zero (0) to one (1) and divided into categories with strictly increasing weights. In one example, the HRR parameter range may be divided into a first category of a zero HRR value and may assign this category a weight of zero; a second category of HRR values falling between zero (0) and the user's anaerobic threshold (AT) and may assign this category a weight of one (1); a third category of HRR values falling between the user's anaerobic threshold (AT) and a threshold at which the user's body employs creatine triphosphate for respiration (CPT) and may assign this category a weight of 18; and a fourth category of HRR values falling between the creatine triphosphate threshold (CPT) and one (1) and may assign this category a weight of 42, although other numbers of HRR categories and different weight values are possible. That is, in this example, the weights are defined as:

$$w ( v ) = \{ \begin{matrix}0 & : v = 0 \\ 1 & : v ∈ ( 0 , AT ] \\ 18 & : v ∈ ( AT , CPT ] \\ 42 & : v ∈ ( CPT , 1 ]\end{matrix}$$

In another exemplary embodiment of the weighting scheme, the HRR time series is weighted iteratively based on the intensity scores determined thus far (e.g., the intensity score accrued thus far) and the path taken by the HRR values to get to the present intensity score. In another exemplary embodiment of the weighting scheme, a predictive approach is used by modeling the weights or coefficients to be the coefficient estimates of a logistic regression model. One of ordinary skill in the art will recognize that two or more aspects of any of the disclosed weighting schemes may be applied separately or in combination in an exemplary method for determining an intensity score.

In one aspect, heart rate zones quantify the intensity of workouts by weighing and comparing different levels of heart activity as percentages of maximum heart rate. Analysis of the amount of time an individual spends training at a certain percentage of his/her MHR may reveal his/her state of physical exertion during a workout. This intensity, developed from the heart rate zone analysis, motion, and activity, may then indicate his/her need for rest and recovery after the workout, e.g., to minimize delayed onset muscle soreness (DOMS) and prepare him/her for further activity. As discussed above, MHR, heart rate zones, time spent above the anaerobic threshold, and HRV in RSA (Respiratory Sinus Arrhythmia) regions—as well as personal information (gender, age, height, weight, etc.) may be utilized in data processing.

A recovery score or indicator provides an accurate indication of the level of recovery of a user's body and health after a period of physical exertion. The human autonomic nervous system controls the involuntary aspects of the body's physiology and is typically subdivided into two branches: parasympathetic (deactivating) and sympathetic (activating). Heart rate variability (HRV), i.e., the fluctuation in inter-heartbeat interval time, is a commonly studied result of the interplay between these two competing branches. Parasympathetic activation reflects inputs from internal organs, causing a decrease in heart rate. Sympathetic activation increases in response to stress, exercise and disease, causing an increase in heart rate. For example, when high intensity exercise takes place, the sympathetic response to the exercise persists long after the completion of the exercise. When high intensity exercise is followed by insufficient recovery, this imbalance lasts typically until the next morning, resulting in a low morning HRV. This result should be taken as a warning sign as it indicates that the parasympathetic system was suppressed throughout the night. While suppressed, normal repair and maintenance processes that ordinarily would occur during sleep were suppressed as well. Suppression of the normal repair and maintenance processes results in an unprepared state for the next day, making subsequent exercise attempts more challenging.

The recovery score is customized and adapted for the unique physiological properties of the user and takes into account, for example, the user's heart rate variability (HRV), resting heart rate, sleep quality and recent physiological strain (indicated, in one example, by the intensity score of the user). In one exemplary embodiment, the recovery score is a weighted combination of the user's heart rate variability (HRV), resting heart rate, sleep quality indicated by a sleep score, and recent strain (indicated, in one example, by the intensity score of the user). In an exemplar, the sleep score combined with performance readiness measures (such as, morning heart rate and morning heart rate variability) provides a complete overview of recovery to the user. By considering sleep and HRV alone or in combination, the user can understand how exercise-ready he/she is each day and to understand how he/she arrived at the exercise-readiness score each day, for example, whether a low exercise-readiness score is a predictor of poor recovery habits or an inappropriate training schedule. This insight aids the user in adjusting his/her daily activities, exercise regimen and sleeping schedule therefore obtain the most out of his/her training.

In some cases, the recovery score may take into account perceived psychological strain experienced by the user. In some cases, perceived psychological strain may be detected from user input via, for example, a questionnaire on a mobile device or web application. In other cases, psychological strain may be determined automatically by detecting changes in sympathetic activation based on one or more parameters including, but not limited to, heart rate variability, heart rate, galvanic skin response, and the like.

With regard to the user's HRV used in determining the recovery score, suitable techniques for analyzing HRV include, but are not limited to, time-domain methods, frequency-domain methods, geometric methods and non-linear methods. In one embodiment, the HRV metric of the root-mean-square of successive differences (RMSSD) of RR intervals is used. The analytics system may consider the magnitude of the differences between 7-day moving averages and 3-day moving averages of these readings for a given day. Other embodiments may use Poincaré Plot analysis or other suitable metrics of HRV.

The recovery score algorithm may take into account RHR along with history of past intensity and recovery scores.

With regard to the user's resting heart rate, moving averages of the resting heart rate are analyzed to determine significant deviations. Consideration of the moving averages is important since day-to-day physiological variation is quite large even in healthy individuals. Therefore, the analytics system may perform a smoothing operation to distinguish changes from normal fluctuations.

Although an inactive condition, sleep is a highly active recovery state during which a major portion of the physiological recovery process takes place. Nonetheless, a small, yet significant, amount of recovery can occur throughout the day by rehydration, macronutrient replacement, lactic acid removal, glycogen re-synthesis, growth hormone production and a limited amount of musculoskeletal repair. In assessing the user's sleep quality, the analytics system generates a sleep score using continuous data collected by an exemplary physiological measurement system regarding the user's heart rate, skin conductivity, ambient temperature and accelerometer/gyroscope data throughout the user's sleep. Collection and use of these four streams of data enable an understanding of sleep previously only accessible through invasive and disruptive over-night laboratory testing. For example, an increase in skin conductivity when ambient temperature is not increasing, the wearer's heart rate is low, and the accelerometer/gyroscope shows little motion, may indicate that the wearer has fallen asleep. The sleep score indicates and is a measure of sleep efficiency (how good the user's sleep was) and sleep duration (if the user had sufficient sleep). Each of these measures is determined by a combination of physiological parameters, personal habits and daily stress/strain (intensity) inputs. The actual data measuring the time spent in various stages of sleep may be combined with the wearer's recent daily history and a longer-term data set describing the wearer's personal habits to assess the level of sleep sufficiency achieved by the user. The sleep score is designed to model sleep quality in the context of sleep duration and history. It thus takes advantage of the continuous monitoring nature of the exemplary physiological measurement systems disclosed herein by considering each sleep period in the context of biologically-determined sleep needs, pattern-determined sleep needs and historically-determined sleep debt.

The recovery and sleep score values are stored on a non-transitory storage medium for retrieval, display and usage. The recovery and/or sleep score values are, in some embodiments, displayed on a user interface rendered on a visual display device. The recovery and/or sleep score values may be displayed as numbers and/or with the aid of graphical tools, e.g., a graphical display of the scale of recovery scores with current score, and the like. In some embodiments, the recovery and/or sleep score may be indicated by audio. The recovery score values are, in some embodiments, displayed along with one or more quantitative or qualitative pieces of information on the user including, but not limited to, whether the user has recovered sufficiently, what level of activity the user is prepared to perform, whether the user is prepared to perform an exercise routine a particular desired intensity, whether the user should rest and the duration of recommended rest, whether the exercise regimen of the user should be automatically adjusted (e.g., made easier if the recovery score is low), and the like. In one embodiment, the analytics system may automatically generate, store and display an exercise regimen customized based on the recovery scores of the user alone or in combination with the intensity scores.

As discussed above, the sleep performance metric may be based on parameters like the number of hours of sleep, sleep onset latency, and the number of sleep disturbances. In this manner, the score may compare a tactical athlete's duration and quality of sleep in relation to the tactical athlete's evolving sleep need (e.g., a number of hours based on recent strain, habitual sleep need, signs of sickness, and sleep debt). By way of example, a soldier may have a dynamically changing need for sleep, and it may be important to consider the total hours of sleep in relation to the amount of sleep that may have been required. By providing an accurate sensor for sleep and sleep performance, an aspect may evaluate sleep in the context of the overall day and lifestyle of a specific user.

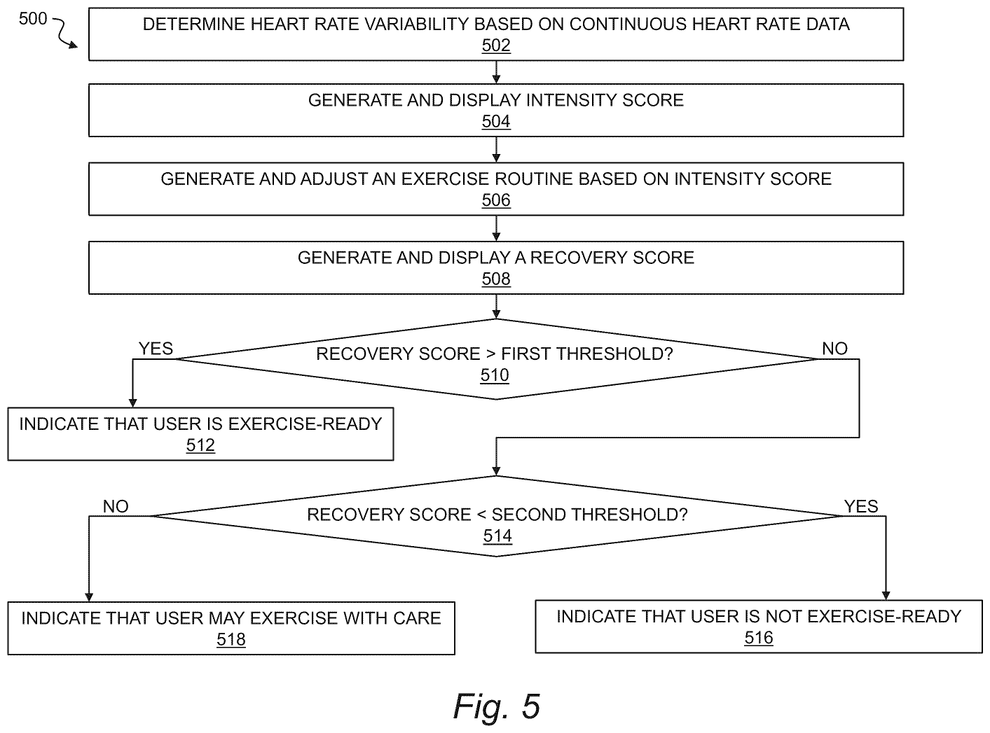

FIG. 5 is a flow chart illustrating a method by which a user may use intensity and recovery scores. As shown in step 502, the method 500 may include the wearable physiological measurement system determining heart rate variability (HRV) measurements based on continuous heart rate data collected by an exemplary physiological measurement system. In some cases, it may take the collection of several days of heart rate data to obtain an accurate baseline for the HRV. As shown in step 504, the method 500 may include the analytics system generating and displaying intensity score for an entire day or an exercise routine. In some cases, the analytics system may display quantitative and/or qualitative information corresponding to the intensity score.

As shown in step 506, the method 500 may include the analytics system automatically generating or adjusting an exercise routine or regimen based on the user's actual intensity scores or desired intensity scores. For example, based on inputs of the user's actual intensity scores, a desired intensity score (that is higher than the actual intensity scores) and a first exercise routine currently performed by the user (e.g., walking), the analytics system may recommend a second different exercise routine that is typically associated with higher intensity scores than the first exercise routine (e.g., running).

As shown in step 508, the method 500 may include, at any given time during the day (e.g., every morning), the analytics system generating and displaying a recovery score. In some cases, the analytics system may display quantitative and/or qualitative information corresponding to the intensity score. For example, as shown in step 510, the method 500 may include the analytics system determining if the recovery is greater than (or equal to or greater than) a first predetermined threshold (e.g., about 60% to about 80% in some examples) that indicates that the user is recovered and is ready for exercise. If this is the case, as shown in step 512, the method 500 may include the analytics system indicating that the user is ready to perform an exercise routine at a desired intensity or that the user is ready to perform an exercise routine more challenging than the past day's routine. Otherwise, as shown in step 514, the method 500 may include the analytics system determining if the recovery is lower than (or equal to or lower than) a second predetermined threshold (e.g., about 10% to about 40% in some examples) that indicates that the user has not recovered. If this is the case, as shown in step 516, the method 500 may include the analytics system indicating that the user should not exercise and should rest for an extended period. The analytics system may, in some cases, the duration of recommended rest. Otherwise, as shown in step 518, the method 500 may include the analytics system indicating that the user may exercise according to his/her exercise regimen while being careful not to overexert him/herself. The thresholds may, in some cases, be adjusted based on a desired intensity at which the user desires to exercise. For example, the thresholds may be increased for higher planned intensity scores.

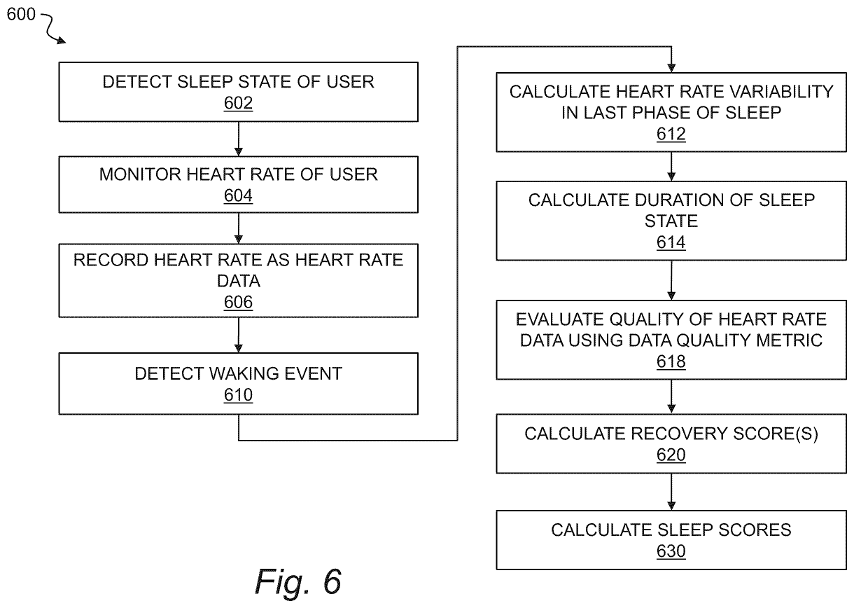

FIG. 6 is a flow chart illustrating a method for detecting heart rate variability in sleep states. The method 600 may be used in cooperation with any of the devices, systems, and methods described herein, such as by operating a wearable, continuous physiological monitoring device to perform the following steps. The wearable, continuous physiological monitoring system may for example include a processor, one or more light emitting diodes, one or more light detectors configured to obtain heart rate data from a user, and one or more other sensors to assist in detecting stages of sleep. In general, the method 600 aims to measure heart rate variability in the last phase of sleep before waking in order to provide a consistent and accurate basis for calculating a physical recovery score.

As shown in step 602, the method 600 may include detecting a sleep state of a user. This may, for example, include any form of continuous or periodic monitoring of sleep states using any of a variety of sensors or algorithms as generally described herein.

Sleep states (also be referred to as “sleep phases,” “sleep cycles,” “sleep stages,” or the like) may include rapid eye movement (REM) sleep, non-REM sleep, or any states/stages included therein. The sleep states may include different phases of non-REM sleep, including Stages 1-3. Stage 1 of non-REM sleep generally includes a state where a person's eyes are closed, but the person can be easily awakened; Stage 2 of non-REM sleep generally includes a state where a person is in light sleep, i.e., where the person's heart rate slows and their body temperature drops in preparation for deeper sleep; and Stage 3 of non-REM sleep generally includes a state of deep sleep, where a person is not easily awakened. Stage 3 is often referred to as delta sleep, deep sleep, or slow wave sleep (i.e., from the high amplitude but small frequency brain waves typically found in this stage). Slow wave sleep is thought to be the most restful form of sleep, which relieves subjective feelings of sleepiness and restores the body.

REM sleep on the other hand typically occurs 1-2 hours after falling asleep. REM sleep may include different periods, stages, or phases, all of which may be included within the sleep states that are detected as described herein. During REM sleep, breathing may become more rapid, irregular and shallow, eyes may jerk rapidly (thus the term “Rapid Eye Movement” or “REM”), and limb muscles may be temporarily paralyzed. Brain waves during this stage typically increase to levels experienced when a person is awake. Also, heart rate, cardiac pressure, cardiac output, and arterial pressure may become irregular when the body moves into REM sleep. This is the sleep state in which most dreams occur, and, if awoken during REM sleep, a person can typically remember the dreams. Most people experience three to five intervals of REM sleep each night.

Homeostasis is the balance between sleeping and waking, and having proper homeostasis may be beneficial to a person's health. Lack of sleep is commonly referred to as sleep deprivation, which tends to cause slower brain waves, a shorter attention span, heightened anxiety, impaired memory, mood disorders, and general mental, emotional, and physical fatigue. Sleep debt (the effect of not getting enough sleep) may result in the diminished abilities to perform high-level cognitive functions. A person's circadian rhythms (i.e., biological processes that display an endogenous, entrainable oscillation of about 24 hours) may be a factor in a person's optimal amount of sleep. Thus, sleep may in general be usefully monitored as a proxy for physical recovery. However, a person's heart rate variability at a particular moment during sleep—during the last phase of sleep preceding a waking event—can further provide an accurate and consistent basis for objectively calculating a recovery score following a period of sleep.

According to the foregoing, sleep of a user may be monitored to detect various sleep states, transitions, and other sleep-related information. For example, the device may monitor/detect the duration of sleep states, the transitions between sleep states, the number of sleep cycles or particular states, the number of transitions, the number of waking events, the transitions to an awake state, and so forth. Sleep states may be monitored and detected using a variety of strategies and sensor configurations according to the underlying physiological phenomena. For example, body temperature may be usefully correlated to various sleep states and transitions. Similarly, galvanic skin response may be correlated to sweating activity and various sleep states, any of which may also be monitored, e.g., with a galvanic skin response sensor, to determine sleep states. Physical motion can also be easily monitored using accelerometers or the like, which can be used to detect waking or other activity involving physical motion. In another aspect, heart rate activity itself may be used to infer various sleep states and transitions, either alone or in combination with other sensor data. Other sensors may also or instead be used to monitor sleep activity, such as brain wave monitors, pupil monitors, and so forth, although the ability to incorporate these types of detection into a continuously wearable physiological monitoring device may be somewhat limited depending on the contemplated configuration.

As shown in step 604, the method 600 may include monitoring a heart rate of the user substantially continuously with the continuous physiological monitoring system. Continuous heart rate monitoring is described above in significant detail, and the description is not repeated here except to note generally that this may include raw sensor data, heart rate data or peak data, and heart rate variability data over some historical period that can be subsequently correlated to various sleep states and activities.

As shown in step 606, the method 600 may include recording the heart rate as heart rate data. This may include storing the heart rate data in any raw or processed form on the device, or transmitting the data to a local or remote location for storage. In one aspect, the data may be stored as peak-to-peak data or in some other semi-processed form without calculating heart rate variability. This may be useful as a technique for conserving processing resources in a variety of contexts, for example where only the heart rate variability at a particular time is of interest. Data may be logged in some unprocessed or semi-processed form, and then the heart rate variability at a particular point in time can be calculated once the relevant point in time has been identified.

As shown in step 610, the method 600 may include detecting a waking event at a transition from the sleep state of the user to an awake state. It should be appreciated that the waking event may be a result of a natural termination of sleep, e.g., after a full night's rest, or in response to an external stimulus that causes awakening prior to completion of a natural sleep cycle. Regardless of the precipitating event(s), the waking event may be detected via the various physiological changes described above, or using any other suitable techniques. While the emphasis herein is on a wearable, continuous monitoring device, it will be understood that the device may also receive inputs from an external device such as a camera (for motion detection) or an infrared camera (for body temperature detection) that can be used to aid in accurately assessing various sleep states and transitions.

Thus the wearable, continuous physiological monitoring system may generally detect a waking event using one or more sensors including, for example, one or more of an accelerometer, a galvanic skin response sensor, a light sensor, and so forth. For example, in one aspect, the waking event may be detected using a combination of motion data and heart rate data.

As shown in step 612, the method 600 may include calculating a heart rate variability of the user at a moment in a last phase of sleep preceding the waking event based upon the heart rate data. While a waking event and a history of sleep states are helpful information for assessing recovery, the method 600 described herein specifically contemplates use of the heart rate variability in a last phase of sleep as a consistent foundation for calculating recovery scores for a device user. Thus, step 612 may also include detecting a slow wave sleep period immediately prior to the waking event, or otherwise determining the end of a slow wave or deep sleep episode immediately preceding the waking event.

It will be appreciated that the last phase of sleep preceding a natural waking event may be slow wave sleep. However, where a sleeper is awakened prematurely, this may instead include a last recorded episode of REM sleep or some other phase of sleep immediately preceding the waking event. This moment—the end of the last phase of sleep before waking—is the point at which heart rate variability data provides the most accurate and consistent indicator of physical recovery. Thus, with the appropriate point of time identified, the historical heart rate data (in whatever form) may be used with the techniques described above to calculate the corresponding heart rate variability. It will be further noted that the time period for this calculation may be selected with varying degrees of granularity depending on the ability to accurate detect the last phase of sleep and an end of the last phase of sleep. Thus for example, the time may be a predetermined amount of time before waking, or at the end of slow wave sleep, or some predetermined amount of time before the end of slow wave sleep is either detected or inferred. In another aspect, an average heart rate variability or similar metric may be determined for any number of discrete measurements within a window around the time of interest.

As shown in step 614, the method 600 may include calculating a duration of the sleep state. The quantity and quality of sleep may be highly relevant to physical recovery, and as such the duration of the sleep state may be used to calculate a recovery score.

As shown in step 618, the method 600 may include evaluating a quality of heart rate data using a data quality metric for a slow wave sleep period, e.g., the slow wave sleep period occurring most recently before the waking event. As noted above, the quality of heart rate measurements may vary over time for a variety of reasons. Thus the quality of heart rate data may be evaluated prior to selecting a particular moment or window of heart rate data for calculating heart rate variability, and the method 600 may include using this quality data to select suitable values for calculating a recovery score. For example, the method 600 may include calculating the heart rate variability for a window of predetermined duration within the slow wave sleep period having the highest quality of heart rate data according to the data quality metric.

As shown in step 620, the method 600 may include calculating a recovery score for the user based upon the heart rate variability from the last phase of sleep. The calculation may be based on other sources of data. For example, the calculation of recovery score may be based on the duration of sleep, the stages of sleep detected or information concerning the stages (e.g., amount of time in certain stages), information regarding the most recent slow wave sleep period or another sleep period/state, information from the GSR sensor or other sensor(s), and so on. The method 600 may further include calculating additional recovery scores after one or more other waking events of the user for comparison to the previously calculated recovery score. The actual calculation of a discovery score is described in substantial detail above, and this description is not repeated here except to note that the use of a heart rate variability measurement from the last phase of sleep provides an accurate and consistent basis for evaluating the physical recovery state of a user following a period of sleep.

As shown in step 630, the method 600 may include calculating a sleep score and communicating this score to a user.

In one aspect, the sleep score may be a measure of prior sleep performance. For example, a sleep performance score may quantify, on a scale of 0-100, the ratio of the hours of sleep during a particular resting period compared to the sleep needed. On this scale, if a user sleeps six hours and needed eight hours of sleep, then the sleep performance may be calculated as 75%. The sleep performance score may begin with one or more assumptions about needed sleep, based on, e.g., age, gender, health, fitness level, habits, genetics, and so forth and may be adapted to actual sleep patterns measured for an individual over time.

The sleep score may also or instead include a sleep need score or other objective metric that estimates an amount of sleep needed by the user of the device in a next sleep period. In general, the score may be any suitable quantitative representation including, e.g., a numerical value over some predetermined scale (e.g., 0-10, 1-100, or any other suitable scale) or a representation of a number of hours of sleep that should be targeted by the user. In another aspect, the sleep score may be calculated as the number of additional hours of sleep needed beyond a normal amount of sleep for the user.

The score may be calculated using any suitable inputs that capture, e.g., a current sleep deficit, a measure of strain or exercise intensity over some predetermined prior interval, an accounting for any naps or other resting, and so forth. A variety of factors may affect the actual sleep need, including physiological attributes such as age, gender, health, genetics and so forth, as well as daytime activities, stress, napping, sleep deficit or deprivation, and so forth. The sleep deficit may itself be based on prior sleep need and actual sleep performance (quality, duration, waking intervals, etc.) over some historical window. In one aspect, an objective scoring function for sleep need may have a model of the form:

$$SleepNeed = Baseline + {f}_{1} ( strain ) + {f}_{2} ( debt ) - Naps$$

In general, this calculation aims to estimate the ideal amount of sleep for best rest and recovery during a next sleep period. When accounting for time falling asleep, periods of brief wakefulness, and so forth, the actual time that should be dedicated to sleep may be somewhat higher, and this may be explicitly incorporated into the sleep need calculation, or left for a user to appropriately manage sleep habits.

In general, the baseline sleep may represent a standard amount of sleep needed by the user on a typical rest day (e.g., with no strenuous exercise or workout). As noted above, this may depend on a variety of factors, and may be estimated or measured for a particular individual in any suitable manner. The strain component, ƒ₁(strain), may be assessed based on a previous day's physical intensity, and will typically increase the sleep need. Where intensity or strain is measured on an objective scale from 0 to 21, the strain calculation may take the following form, which yields an additional sleep time needed in minutes for a strain, i:

$$f ( i ) = \frac{1.7}{1 + {e}^{\frac{17 - i}{3.5}}}$$

The sleep debt, ƒ₂(debt), may generally measure a carryover of needed sleep that was not attained in a previous day. This may be scaled, and may be capped at a maximum, according to individual sleep characteristics or general information about long term sleep deficit and recovery. Naps may also be accounted for directly by correcting the sleep need for any naps that have been taken, or by calculating a nap factor that is scaled or otherwise manipulated or calculated to more accurately track the actual effect of naps on prospective sleep need.

However calculated, the sleep need may be communicated to a user, such as by displaying a sleep need on a wrist-worn physiological monitoring device, or by sending an e-mail, text message or other alert to the user for display on any suitable device.

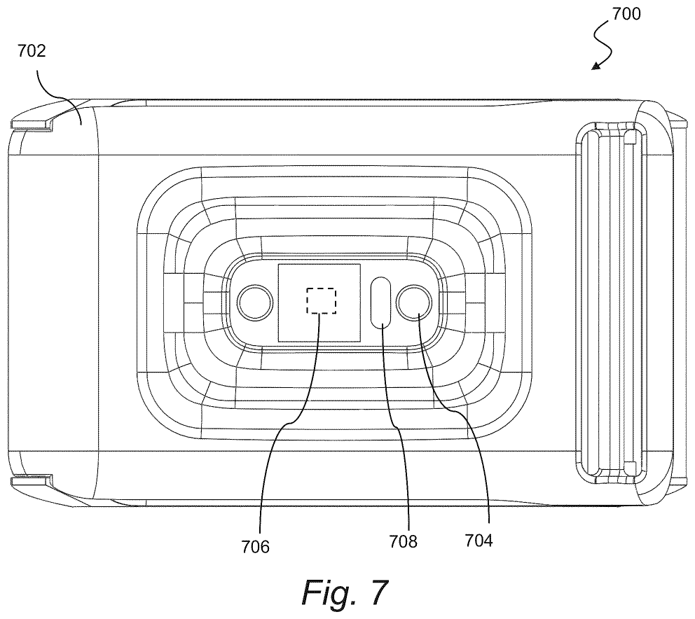

FIG. 7 is a bottom view of a wearable, continuous physiological monitoring device (the side facing a user's skin). As shown in the figure, the wearable, continuous physiological monitoring system 700 includes a wearable housing 702, one or more sensors 704, a processor 706, and a light source 708.

The wearable housing 702 may be configured such that a user can wear a continuous physiological monitoring device as part of the wearable, continuous physiological monitoring system 700. The wearable housing 702 may be configured for cooperation with a strap or the like, e.g., for engagement with an appendage of a user.

The one or more sensors 704 may be disposed in the wearable housing 702. In one aspect, the one or more sensors 704 include a light detector configured to provide data to the processor 706 for calculating a heart rate variability. The one or more sensors 704 may also or instead include an accelerometer configured to provide data to the processor 706 for detecting a sleep state or a waking event. In an implementation, the one or more sensors 704 measure a galvanic skin response of the user.

The processor 706 may be disposed in the wearable housing 702. The processor 706 may be configured to operate the one or more sensors 704 to detect a sleep state of a user wearing the wearable housing 702. The processor 706 may be further configured to monitor a heart rate of the user substantially continuously, and to record the heart rate as heart rate data without calculating a heart rate variability for the user. The processor 706 may also or instead be configured to detect a waking event at a transition from the sleep state of the user to an awake state, and to calculate the heart rate variability of the user at a moment in the last phase of sleep preceding the waking event based upon the heart rate data. The processor 706 may further be configured to calculate a recovery score for the user based upon the heart rate variability from the last phase of sleep.

The light source 708 may be coupled to the wearable housing 702 and controlled by the processor 706. The light source 708 may be directed toward the skin of a user's appendage. Light from the light source 708 may be detected by the one or more sensors 704.

Physiological signals acquired using the various different sensors described herein can be sensitive to conditions under which the physiological signal is obtained. Thus, for example, the physiological signal obtained during certain activities and/or under certain conditions can contain significant amounts of noise, or may have characteristics that vary according to the type of activity or other physical or physiological context. Accordingly, where it is desirable to continuously monitor a physiological signal, it can be advantageous to process the signal using the following techniques in order to reduce or eliminate the negative effects of confounding factors such as motion of the wearable, type of activity, the physical interface with a wearer's skin, weather or other ambient conditions, and so forth.

### Pulse Shape Analysis

The shape of a PPG signal contains more information than just heart rate. In a young healthy subject, for example, the pulse usually starts with a sharp rise, a first big peak for systolic cycles, a second smaller peak for diastolic cycles, and finally the descent to the baseline. Each feature of the PPG pulse and the relation between different features carry some information about cardiovascular health. For example, the fast-rising slope at the start of the pulse may be correlated with strong heart muscle while the weak second peak may be an indication of bad circulation. Unlike HR and HRV, a single pulse may provide information to determine the recovery, strain, or cardiovascular age without calibration. The pulse shape may also be used for sleep staging, scoring recovery, and/or performance optimization.

Suitable methods for analyzing PPG pulse waves are described in the following references: Liu, Prediction of Physiological Metrics from Machine Learning Models on PPG Pulse Shapes, 2020; Elgendi, On the analysis of fingertip photoplethysmogram signals. Current Cardiology Reviews, 8(1): 12, 2012; Kamal et al., Skin photoplethysmography-a review. Computer Methods and Programs in Biomedicine, 28:257-269, 1989; Hertzman, Photoelectric plethysmography of the fingers and toes in man. Experimental Biology and Medicine, 1937; Marcin, Diastole vs. systole: A guide to blood pressure, https://www.healthline.com/health/diastole-vs-systole, 2019; Takazawa et al., Assessment of vasoactive agents and vascular aging by the second derivative of photoplethysmogram waveform, Hypertension 32(2): 365-370, 1998; Ahn, New aging index using signal features of both photoplethysmograms and acceleration plethysmograms. Healthcare Informatics Research, 23(1), 2017; Elgendi, Standard terminologies for photoplethysmogram signals, Current Cardiology Reviews, 8(3): 215-219, August 2012; Imanaga et al., Correlation between wave components of the second derivative of plethysmogram and arterial distensibility, Japanese Heart Journal, 39(6): 775-784, 1998; Back et al., Second derivative of photoplethysmography for estimating vascular aging, pages 70-72, 2007; Usman, Second derivative of photoplethysmogram in estimating vascular aging among diabetic patients, pages 1-3, 2009; Khalid et al., Blood pressure estimation using photoplethysmography only: Comparison between different machine learning approaches, Journal of Healthcare Engineering, 2018; Liang et al., Photoplethysmography and deep learning: Enhancing hypertension risk stratification, Smart Biomedical Sensors, 2018; Berryhill et al., Effect of wearables on sleep in healthy individuals: a randomized crossover trial and validation study, Journal of Clinical Sleep Medicine, 16(5): 775-783, 2020; T. pandas development team, pandas-dev/pandas: Pandas, February 2020; Oliphant, A guide to NumPy, volume 1, Trelgol Publishing USA, 2006; Van Der Walt et al., The numpy array: a structure for efficient numerical computation, Computing in Science & Engineering, 13(2): 22, 2011; Hunter, Matplotlib: A 2d graphics environment, Computing in Science & Engineering, 9(3): 90-95, 2007; Pedregosa et al., Scikit-learn: Machine learning in Python, Journal of Machine Learning Research, 12:2825-2830, 2011; Abadi et al., TensorFlow: Large-scale machine learning on heterogeneous systems, 2015; Geron, Hands-On Machine Learning with Scikit-Learn and TensorFlow: Concepts, Tools, and Techniques to Build Intelligent Systems, O'Reilly Media, Inc., 1st edition, 2017; Abdi, Partial least squares regression and projection on latent structure regression (pls regression), WIREs Computational Statistics, 2(1): 97-106, January 2010. The entire content of these references is hereby incorporated by reference.

While these references suggest the wealth of available information in cardiac pulse signals, they appear to focus on static analysis of a single pulse shape, and they are based on very small numbers of samples. By contrast, the applicant has spent years acquiring pulse samples from wearers of physiological monitoring devices, and now has a library of millions of pulse samples from thousands of users, all accurately labelled with objective data about the users. This advantageously enables the creation of accurate machine learning models that can be trained and tested over very large data sets covering a wide range of users and a wide range of user conditions. The resulting models can improve on the static analysis techniques described above, and further facilitate dynamic analysis of changes in pulse shape, e.g., from pulse to pulse for a particular user or among pulses for different users, based on a latent space representation of pulse shape developed for the machine learning model.

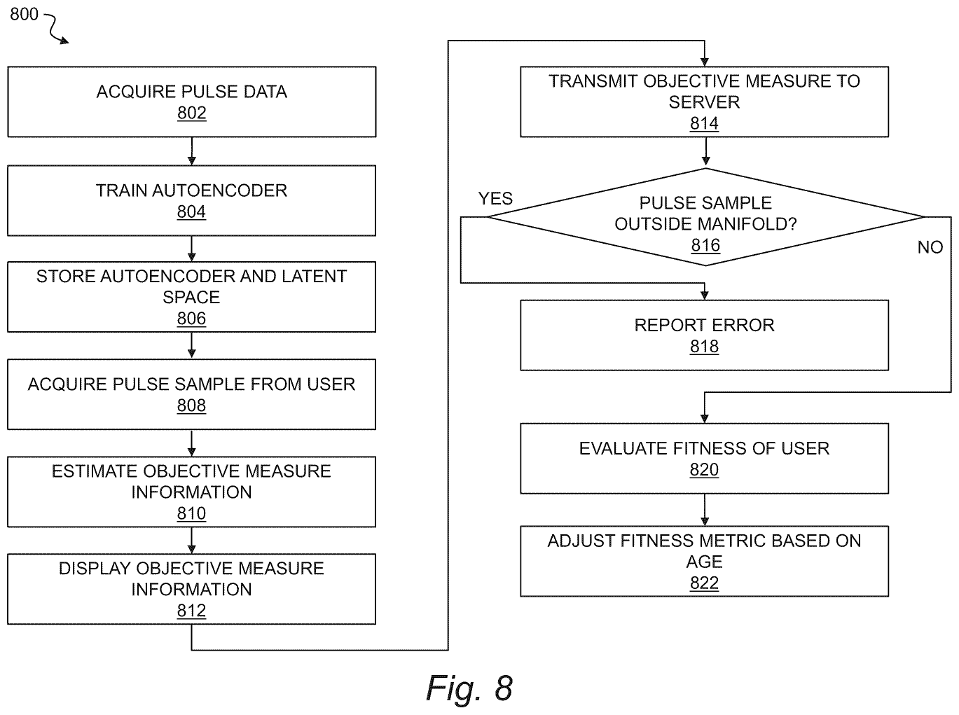

FIG. 8 is a flow chart illustrating a method for estimating an objective measure with pulse data using a machine learning model. A machine learning model for analysis of PPG pulse waves may benefit significantly from large data sets. For example, the use of a large data set may facilitate the construction of accurate predictive models for an objective measure of an individual user such as cardiovascular fitness or age. In general, a machine learning model may encode pulse data into a latent space with generated features. The latent space may then give an estimate of the value of the objective measure of an individual.

As shown in step 802, the method 800 may include acquiring pulse data including a plurality of heart pulse samples. PPG data of heart pulses may be acquired from individuals and separated into a training set and a testing set. As an example, the training and testing sets may be taken from a data set that includes millions of pulse shapes from thousands of individuals aged 18 to 70, although other population demographics may be used. The training and testing sets may be labelled with recorded objective measures of every individual represented in the training and testing sets, such as age, height, weight, gender, and so forth.

As shown in step 804, the method 800 may include training an autoencoder to encode the plurality of heart pulse samples into a latent space that differentiates among the plurality of heart samples according to an objective measure. The autoencoder may be any machine learning model that encodes pulse shapes to a latent space. For example, the autoencoder may be a feed-forward neural network having three fully-connected hidden layers and a 4-D latent space. While more or less layers may be used, and the latent space may have more or less dimensions, this model has been demonstrated to accurately and efficiently encode pulse shape for the purposes described herein. During training, the representation of the heart pulse samples in latent space may be labelled with a corresponding objective measure of a subject associated with the one of the plurality of heart pulse samples. The autoencoder may then be trained over 50 epochs to optimize the mean squared error between the input, an original pulse waveform, and the output, a reconstructed pulse waveform.

As shown in step 806, the method 800 may include storing a representation of the autoencoder and the latent space as a machine learning model. A 4-D model may advantageously provide a compact form that can be locally deployed for use, e.g., in a memory of a wearable physiological monitoring device, which may include any of the wearable devices described herein. However, the model may also or instead be deployed at a remote location such as a user computer, a user cellphone, or a remote server, any of which may receive a pulse shape and process the pulse shape using the machine learning model as described herein. In another aspect, an optimized or compressed version of a machine learning model may be deployed locally, e.g., on the wearable device, and a full machine learning model may be remotely accessible so that results can be quickly obtained on the local device while more accurate results can be obtained remotely if/when desired. Thus in general, the storing and processing of data as described herein may be accomplished using the wearable physiological monitoring device, a device in communication with the wearable physiological monitoring device, or a combination of the foregoing. In certain aspects, the wearable physiological monitoring device may be used to acquire data, while the storing, processing, and the like may be accomplished using other devices including local devices and/or remote resources accessible through a network.

As shown in step 808, the method 800 may include acquiring a pulse sample from a user with the wearable physiological monitor. The pulse sample may be collected by a wearable physiological measurement system that uses a PPG sensor to continuously collect physiological data from the user. This may be the same device that stores and applies the machine learning model, or this may be a separate device dedicated to data acquisition.

As shown in step 810, the method 800 may include estimating objective measure information for the user with the machine learning model based on the pulse sample. The objective measure may include one or more of a biological age, a cardiovascular age, a fitness level, a blood pressure level, and others. An estimate of an objective measure may be beneficial to a user by informing the user of their own cardiovascular condition and lifestyle. The user may then choose to adjust their lifestyle based on the objective measure in order to improve their wellbeing and health.

To estimate the value of the objective measure for a pulse sample, the autoencoder may output a latent space with generated features based on pulse samples. Within the latent space, the objective measure values of representations nearby in the latent space may be examined. The mean or median of the objective measure values of representations within a radius ball around the sample may be taken as the predictive value of the objective measure value. As an example, a radius of 0.1 in Euclidean distance may be chosen. However, finding all the neighboring points in a ball can be computationally difficult. Instead of using the entire latent space, a random sample of the latent space may be used. For example, in a 2-million-point latent space, 500,000 points or less may be randomly chosen. A k-dimensional tree or other space partitioning structure may then be built from the random sample and used to find neighboring points in the latent space.

As shown in step 812, the method 800 may include displaying the objective measure information to the user. The information may be displayed on a display on the wearable physiological measurement system. The information may also be displayed with a recommendation or alert to the user based on the objective measure information. For example, the objective measure information be an estimate of a cardiovascular age of the user. If the estimate is very high, the display may alert to user and make a recommendation to the user to make a lifestyle change.

As shown in step 814, the method 800 may include transmitting the objective measure to a remote server. The objective measure may be transmitted to a remote server from the wearable physiological measurement system for insertion into a large data set. The updated data set may then be used for further analysis and finetuning of the autoencoder.

As shown in step 816, the method 800 may include comparing the objective measure to a reported objective measure for the user. The user may directly input the reported objective measure into the wearable physiological measurement system. Alternatively, the wearable physiological measurement system may access a user database to obtain previous information for the user. The objective measure may also or instead be calculated based on prior user data. For example, where the user has previously provided a birth date, e.g., for a user account stored on a remote server, this may be used to calculate an age for the user without requiring direct reporting from the user.

As shown in step 818, the method 800 may include reporting an error for a device acquiring the pulse sample when a representation of the pulse sample in the latent space lies outside a predetermined manifold for the objective measure. The objective measure may have predetermined boundaries within the latent space that define possible values for the objective measure. A representation of a pulse sample that falls outside those boundaries may indicate an error with the device's hardware, software, corrupted data, and the like. In another aspect, detecting an error may be based on the probability of a particular pulse shape given a distribution of pulse shapes for an individual user, and/or a population of users.

As shown in step 820, the method 800 may include evaluating a fitness of the user. In an exemplary case, the objective measure may be an age of the user, and the fitness may be evaluated based on a difference between an age estimated by the machine learning model and a reported age provided by the user. For example, predicted age that is younger than the reported age may indicate a high fitness level, whereas an older predicted age may indicate a low fitness level. If the difference is above a predetermined threshold and the predicted age is older than the reported age, a warning may be displayed to the user.

As shown in step 822, the method 800 may include adjusting a fitness metric for the user based on the objective measure. The fitness metric may include one or more of a resting heart rate, maximum heart rate, a sleep score, a recovery score, a strain score, and others. A fitness metric for the user may be more informative if compared to users of a similar demographic and age. Therefore, the fitness metric may be adjusted based on the predicted objective measure information of the user. For example, where the objective measure information is a predicted age of the user, the maximum heart rate may be selected based on the predicted age, or adjusted toward a maximum heart rate for the predicted age. Thus for example, a user with an older cardiovascular age than actual age may have a maximum heart rate adjusted downward to ensure that fitness recommendations, strain calculations, and the like are based on the cardiovascular age of the user (e.g., the actual fitness level) rather than the numerical age.

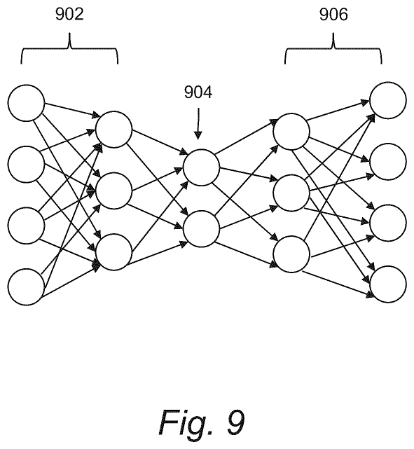

FIG. 9 shows an exemplary autoencoder and decoder. The encoder 902 may be used to create a latent space mapping for pulse shape. For example, a useful latent space mapping for determining age based on pulse shape was constructed using a three second PPG signal captured every fifteen minutes from fifty thousand users and collected over the period of one month, however other similarly large data sets may also or instead be used. In this example, one pulse period was extracted from each three-second data sample and normalized to the maximum value of the pulse. This dataset was used to train an autoencoder network. To simplify an encoder implementation for a wearable physiological monitor, a neural network structure was used, with one hundred inputs, three hidden layers (with fifty outputs, twenty-five outputs, and ten outputs, respectively), and four outputs (e.g., a four dimensional latent space). The decoder 906, which may process the latent space output of the encoder into reconstructed pulse shapes, was implemented using a three-layer convolutional neural network with fifteen nodes each and a one-hundred dimensional output.

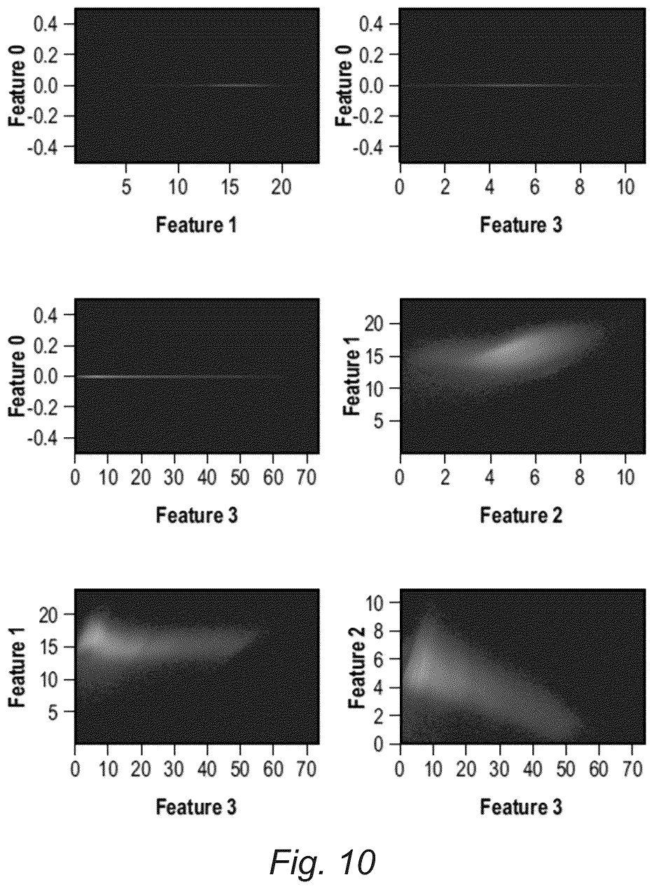

FIG. 10 illustrates exemplary latent spaces for pulse shape created using the data set described above. Each 4-D latent space may be labelled with objective measure information to determine correlations between automatically generated features in the latent space and the objective measure. In this case, the objective measure is the age of an individual. In FIG. 10, the lighter shades represent older ages while the darker shades represent younger ages. There is a noticeable separation of shading within the latent space, suggesting a notable correlation between the generated features and age.

Using the same general process, an athleticism map may be constructed, or any other mapping in latent space that provides an objective measure useful for analyzing physiological data. For example, a latent map may be constructed for overall strain and recovery, sleep staging, changes in fitness or health over time, and so forth. Any resulting encoder map may then be implemented on a wearable physiological monitor or elsewhere to report corresponding tensors for pulse shape at any suitable interval, e.g., every second, every minute, every hour, every day, and so forth, and/or to analyze a current pulse shape to extract corresponding information concerning the objective measure.

In general, a similarity (distance) metric between two pulses in a latent space may be used to drive other features and analysis. The distance metric may, for example, be a Euclidean distance in latent space, or any other metric suitable for measuring similarity based on distance in an n-dimensional space. The main property of the distance metric (dis [PS]) is the preservation of weak continuity:

$$∃ δ , σ for {δ}_{i} , {σ}_{i} ; ∀ 0 < {δ}_{i} < delta ; 0 < {δ}_{i} < sigma \\ ∀ PS ( p ) , PS ( q ) ∈ Dataset if \vert PS ( p ) - PS ( q ) \vert < {σ}_{i} \\ \vert dis ( PS ( p ) , PS ( q ) ) \vert < {δ}_{i} PS ( i ) - PS ( j ) \vert = 0 \text{<=>} dis ( PS ( i ) , PS ( j ) )$$

Using an appropriate latent space and a suitable distance metric, a variety of calculations and inferences may be made based on an incoming stream of physiological pulses for a user. For example, a distance between two pulses in a suitably mapped latent space may be used to calculate a fitness score for a user. Although age has been extensively studied, other objective measures such as sleep, fitness, and strain, appear to be similarly mappable to a latent space that facilitates distance-based analysis among pulses.

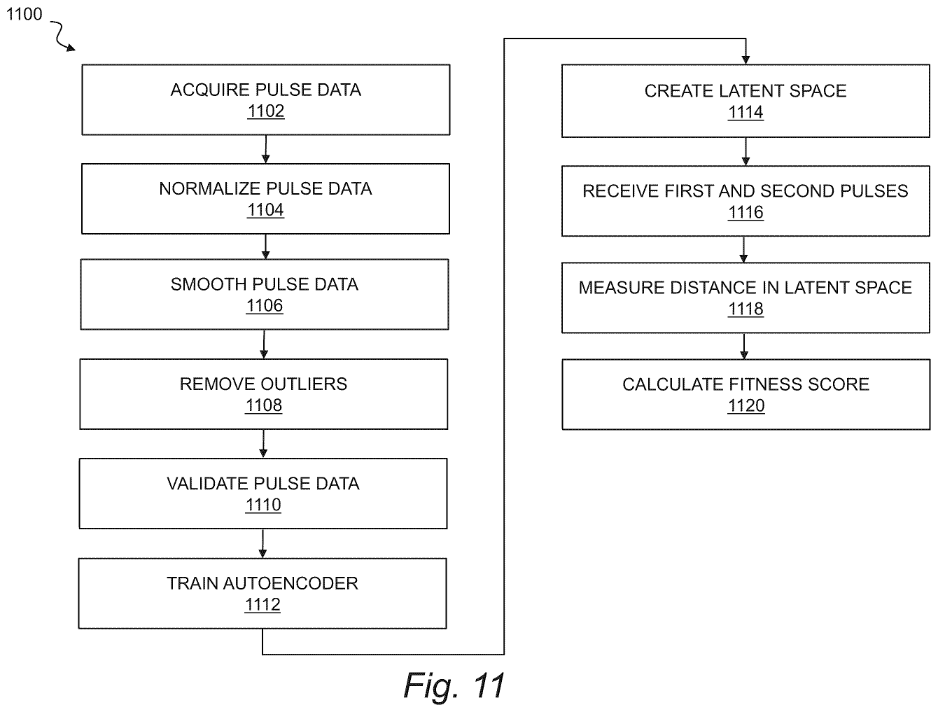

FIG. 11 is a flow chart illustrating a method for calculating a fitness level of a user using pulse data. While other methods of estimating objective measure information use a static pulse shape, useful information may also be obtained from variations in shape between different pulses. A latent space that quantifies pulse shape as one or more features can facilitate side-by-side comparison of different pulses in a manner that permits inferences about fitness (or more specifically, an objective measure of fitness), e.g., based on a comparison of multiple pulses from a single user over short and/or long time intervals, and/or based on measurements during different activities or the same activity.

As shown in step 1102, the method 1100 may include acquiring pulse data including a plurality of heart pulse samples. The pulse data may be acquired from a large pulse data set derived from a large population of individuals using a PPG sensor. The pulse data may also include heart pulse samples from the user.

As shown in step 1104, the method 1100 may include normalizing the pulse data to a maximum pulse value or a maximum magnitude of the pulse signal within each pulse sample. The data set may for example be normalized to values between 0 and 1, with 1 assigned to the maximum pulse value (e.g., the pulse peak), or to any other value useful for comparison among pulses.

As shown in step 1106, the method 1100 may include smoothing the pulse data. The pulse data may be smoothed to distinguish changes from normal fluctuations. Smoothing the pulse data may include calculating a three second rolling mean, or otherwise smoothing or filtering the data to remove spurious or potentially spurious data points.

As shown in step 1108, the method 1100 may include removing outliers in the pulse data. Due to the easily corruptible nature of pulse data for a wearable physiological monitor, it is common for noise to periodically overwhelm the physiological data, e.g., when the monitor strikes a solid object or the user moves so rapidly that contact between the monitor and the skin is interrupted. Outliers in the pulse data may be removed by assessing the z score of peak values (e.g., the a peak, b peak, c peak, and d peak in a heart signal). Individual values may, for example, be considered outliers if they are beyond two, three, or four standard deviations from the mean.

As shown in step 1110, the method 1100 may include validating the pulse data based on pulse features within the pulse signal. For example, seven features are commonly used to characterize a heart signal, and may be used to assess whether a pulse sample should be kept for further analysis: the systolic peak, dicrotic notch, diastolic peak, a peak, b peak, c peak, and d peak. In one aspect, the following validation criteria may be applied to determine whether a particular pulse sample should be retained: (1) the signal's autocorrelation is high, (2) the difference in locations of the diastolic and systolic peaks is not greater than a threshold, (3) the diastolic peak value is greater than half of the signal's highest amplitude, and (4) the dicrotic notch is in the first half of the signal. However, other validation rules and filters may also or instead be used based on a generalized model for an expected pulse shape.

As shown in step 1112, the method 1100 may include applying the pulse data to train an autoencoder network on the latent space. As aforementioned, the autoencoder may be trained to map pulse data into a latent space with automatically generated features.

As shown in step 1114, the method 1100 may include creating a latent space using the pulse data that has been pre-processed as described above. The inputs may include time series data in pulse samples and/or any of the features of a characteristic pulse shape described above, or any other derived quantities or metrics that might usefully be employed to describe pulse shape in a manner that facilitates discrimination among different pulses with respect to an objective measure of interest. While a machine learning model like an autoencoder may be used on raw source data, features may also or instead be extracted manually by analyzing points and lines on pulse waveforms and their derivatives. For example, feature of interest for a heart pulse signal may include one or more of a b/a ratio, a c/a ratio, a d/a ratio/and an e/a ratio. These features may be extracted for a characteristic pulse shape of the pulse data that is obtained, e.g., by averaging pulse shape data for the entire data set, or some other data set that reflects a relevant population of users and/or the history of an individual user. Each feature of the average pulse shape may have an average or median value within the pulse data that may be used as a basis of comparison for evaluating a new sample with the autoencoder.

As shown in step 1116, the method 1100 may include receiving a first pulse of heart rate data and a second pulse of heart rate data from a wearable physiological monitor worn by a user during a window corresponding to the characteristic pulse shape. As aforementioned, pulse data may be acquired using a PPG sensor on a wearable physiological measurement system, or using any other monitoring system or technology corresponding to the source data for the machine learning model. In general, this may include receiving the pulse data on the wearable physiological measurement system, or at a remote resource that receives data from the wearable monitor through a data network. The window for receiving pulse data may be any window of a predetermined time period selected to correspond to the characteristic pulse shape within the pulse data. A first and second pulse occurring during this window (or more precisely, two separate instances of the window) may then be recorded and encoded into the latent space. The second pulse may also or instead be an average pulse shape for a population of users or for a history of the individual user, which may be used as a basis for comparison and analysis within the latent space.

As shown in step 1118, the method 1100 may include measuring a distance within the latent space between the first pulse and the second pulse. The distance may be measured as a Euclidean distance within the latent space, or using any other measure of multi-dimensional distance suitable for analyzing pulses in the latent space.

As shown in step 1120, the method 1100 may include calculating a fitness score for the user based on the distance within the latent space. A fitness score indicative of some aspect of fitness of the user may be correlated with the distance between the first and second pulses. The fitness score may, for example, measure the fitness level of a user by measuring one or more transient parameters specific to the user that are expected to change from day to day such as sleep, strain, recovery, and the like. The fitness score may also or instead measure a descriptive parameter indicative of a general or long-term fitness state relative to a population such as cardiovascular age or cardiovascular fitness.

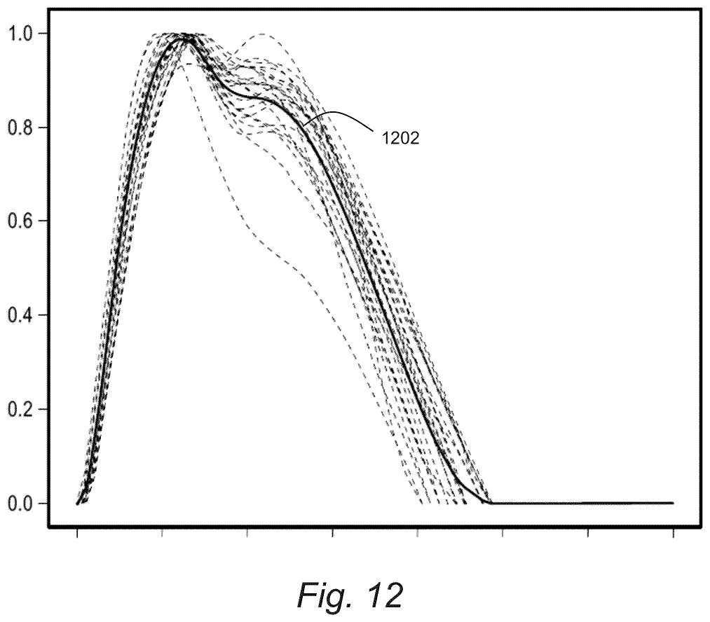

FIG. 12 illustrates an exemplary data set for training an autoencoder. Each pulse shape, recorded by a PPG sensor, is denoted by a plotted curve, with the bolded curve 1202 being a characteristic pulse shape or average pulse shape within the data set. The pulse shapes are normalized to range of zero to one, and have a notable waveform with a systolic peak, followed by a diastolic notch, and a diastolic peak.

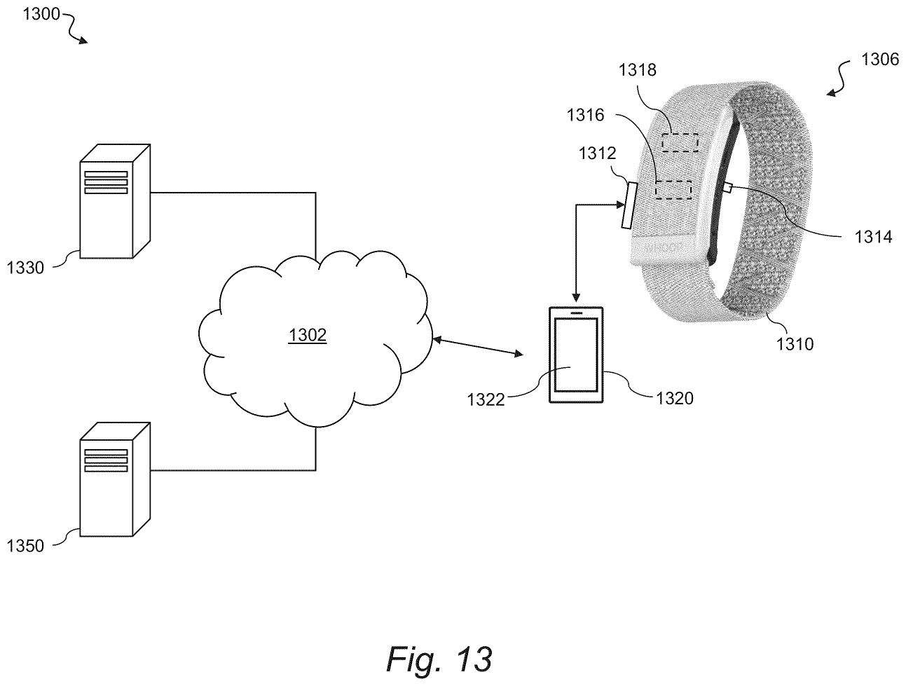

FIG. 13 illustrates a physiological monitoring system, which may be used with the methods described above, or any of the other methods or devices described herein. In general, the system 1300 may include a physiological monitor 1306, a user device 1320, a remote server 1330 with a remote data processing resource (such as any of the processors or processing resources described herein), and one or more other resources 1350, all of which may be interconnected through a data network 1302.

The data network 1302 may be any of the data networks described herein. For example, the data network 1302 may be any network(s) or internetwork(s) suitable for communicating data and information among participants in the system 1300. This may include public networks such as the Internet, private networks, telecommunications networks such as the Public Switched Telephone Network or cellular networks using third generation (e.g., 3G or IMT-2000), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 1300. This may also include local or short range communications networks suitable, e.g., for coupling the physiological monitor 1306 to the user device 1320, or otherwise communicating with local resources.

The physiological monitor 1306 may, in general, be any physiological monitoring device, such as any of the wearable monitors or other monitoring devices described herein, and may include a network interface 1312, one or more sensors 1314, a processor 1316, a memory 1318, and a wearable strap 1310 for retaining the device in a desired location on a user. The network interface 1312 may be configured to coupled one or more participants of the system 1300 in a communicating relationship, e.g., with the remote server 1330. The one or more sensors 1314 may include any of the sensors described herein, or any other sensors suitable for physiological monitoring. By way of example and not limitation, the one or more sensors 1314 may include one or more of a light source, and optical sensor, an accelerometer, a gyroscope, a temperature sensor, a galvanic skin response sensor, an environmental sensor (e.g., for measuring ambient temperature, humidity, lighting, and the like), a geolocation sensor, a temporal sensor, an electrodermal activity sensor, and the like.

The processor 1316 and memory 1318 may be any of the processors and memories described herein and suitable for deployment in a physiological monitoring device. In one aspect, the memory 1318 may store an autoencoder and a latent space as a machine learning model configured to estimate an objective measure based on a pulse sample. The processor 1316 may be configured to receive the pulse sample, to encode the pulse sample with the autoencoder, and to estimate an objective measure of the user based on a location of the pulse sample in the latent space. Alternatively, the memory 1318 may store a latent space for an autoencoder that encodes a number of features of a photoplethysmography pulse signal based on a characteristic pulse shape of photoplethysmography pulse samples of a population, wherein the one or more features include a fitness level associated with a pulse sample. The processor 1316 may then be configured to receive a first pulse sample and a second pulse sample, to encode the first pulse sample and the second pulse sample with the autoencoder into the latent space, to measure a distance within the latent space between the first pulse and the second pulse, and to calculate the fitness score for the user based on the distance within the latent space.

The system 1300 may further include a remote data processing resource executing on a remote server 1330. The remote data processing resource may be any of the processors described herein, and may be configured to receive data transmitted from the memory 1318 of the physiological monitor 1306, and to perform any of the methods described above.

The system 1300 may also include one or more user devices 1320, which may work together with the physiological monitor, e.g., to provide a display for user data and analysis, and/or to provide a communications bridge from the network interface 1312 of the physiological monitor 1306 to the data network 1302 and the remote server 1330. For example, physiological monitor 1306 may communicate locally with a user device 1320, such as a smartphone of a user, via short-range communications, e.g., Bluetooth, or the like, e.g., for the exchange of data between the physiological monitor 1306 and the user device 1320, and the user device 1320 may communicate with the remote server 1330 via the data network 1302. Computationally intensive processing may be performed at the remote server 1330, which may have greater memory capabilities and processing power than the physiological monitor 1306 that acquires the data.

The user device 1320 may include any computing device as described herein, including without limitation a smartphone, a desktop computer, a laptop computer, a network computer, a tablet, a mobile device, a portable digital assistant, a cellular phone, a portable media or entertainment device, and so on. The user device 1320 may provide a user interface 1322 for access to data and analysis by a user, and/or to control operation of the physiological monitor 1306. The user interface 1322 may be maintained by a locally-executing application on the user device 1320, or the user interface 1322 may be remotely served and presented on the user device 1320, e.g., from the remote server 1330 or the one or more other resources 1350.

In general, the remote server may include data storage, a network interface, and/or other processing circuitry. The remote server 1330 may process data from the physiological monitor and perform pulse data analysis or any of the other analyses described herein, and may host a user interface for remote access to this data, e.g., from the user device 1320. The remote server 1330 may include a web server or similar front end that facilitates web-based access by the user devices 1320 or the physiological monitor 1306 to the capabilities of the remote server 1330 or other components of the system 1300.

The other resources 1350 may include any resources that can be usefully employed in the devices, systems, and methods as described herein. For example, these other resources 1350 may include without limitation other data networks, human actors (e.g., programmers, researchers, annotators, editors, analysts, and so forth), sensors (e.g., audio or visual sensors), data mining tools, computational tools, data monitoring tools, algorithms, and so forth. The other resources 1350 may also or instead include any other software or hardware resources that may be usefully employed in the networked applications as contemplated herein. For example, the other resources 1350 may include payment processing servers or platforms used to authorize payment for access, content, or option/feature purchases, or otherwise. In another aspect, the other resources 1350 may include certificate servers or other security resources for third-party verification of identity, encryption or decryption of data, and so forth. In another aspect, the other resources 1350 may include a desktop computer or the like co-located (e.g., on the same local area network with, or directly coupled to through a serial or USB cable) with a user device 1320, wearable strap 1310, or remote server 1330. In this case, the other resources 1350 may provide supplemental functions for components of the system 1300.

The other resources 1350 may also or instead include one or more web servers that provide web-based access to and from any of the other participants in the system 1300. While depicted as a separate network entity, it will be readily appreciated that the other resources 1350 (e.g., a web server) may also or instead be logically and/or physically associated with one of the other devices described herein, and may, for example, include or provide a user interface 1322 for web access to a remote server 1330 or a database in a manner that permits user interaction through the data network 1302, e.g., from the physiological monitor 1306 or the user device 1320.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. For example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A computer program product comprising computer executable code embodied in a non-transitory computer readable medium that, when executing on one or more computing devices, causes the one or more computing devices to perform the steps of:
storing a latent space for photoplethysmography data, the latent space differentiating among heart pulse samples in the photoplethysmography data according to a cardiovascular age associated with each of the heart pulse samples;
acquiring a photoplethysmography signal for a user from a wearable physiological monitor;
identifying a pulse of heart rate data in the photoplethysmography signal for the user;
transforming the pulse of heart rate data into the latent space with an autoencoder;
estimating the cardiovascular age for the user based on a location of the pulse of heart rate data in the latent space; and
presenting the cardiovascular age to the user.

2. The computer program product of claim 1, wherein the cardiovascular age includes a relative cardiovascular age for the user.

3. The computer program product of claim 1, wherein the cardiovascular age includes the cardiovascular age relative to a population of users.

4. The computer program product of claim 1, wherein the cardiovascular age includes a chronological age.

5. The computer program product of claim 1, wherein estimating the cardiovascular age for the user includes calculating the age based on a distance in the latent space between the location of the pulse of heart rate data and a second position of one or more other pulses for one or more different individuals encoded in the latent space.

6. The computer program product of claim 1, further comprising code that performs the step of evaluating a fitness of the user based on a difference between the cardiovascular age for the user and a chronological age reported by the user.

7. The computer program product of claim 1, further comprising code that performs the step of adjusting a maximum heart rate for the user based on the cardiovascular age, and calculating a fitness metric for the user based on the adjusted maximum heart rate.

8. The computer program product of claim 1, wherein a latent space mapping for the latent space is created by:
acquiring a three second sample every fifteen minutes for a plurality of users,
extracting a single pulse from each three second sample for use in training, and
training a network for the autoencoder using the extracted, single pulses and corresponding age data.

9. A method, comprising:
storing a latent space for heart rate data, the latent space differentiating among heart pulse samples in the heart rate data according to a cardiovascular age associated with each of the heart pulse samples;
acquiring a heart rate signal for a user from a wearable physiological monitor;
identifying a pulse of heart rate data in the heart rate signal for the user;
transforming the pulse of heart rate data into the latent space with an autoencoder;
estimating the cardiovascular age for the user based on a location of the pulse of heart rate data in the latent space; and
presenting the cardiovascular age to the user.

10. The method of claim 9, wherein the wearable physiological monitor includes a wrist-worn monitor.

11. The method of claim 9, wherein the cardiovascular age includes a chronological age.

12. The method of claim 9, wherein the cardiovascular age includes a relative cardiovascular age for the user.

13. The method of claim 9, wherein the cardiovascular age includes the cardiovascular age relative to a population of users.

14. The method of claim 9, wherein estimating the cardiovascular age for the user includes calculating the age based on a distance in the latent space between the location of the pulse of heart rate data and a second position of one or more other pulses for one or more different individuals encoded in the latent space.

15. The method of claim 9, further comprising evaluating a fitness of the user based on a difference between the cardiovascular age for the user and a chronological age reported by the user.

16. The method of claim 9, wherein the latent space is created with a data set corresponding to a relevant population for the user.

17. The method of claim 9, wherein the latent space is created with pulse data labeled according to gender.

18. The method of claim 9, further comprising adjusting a maximum heart rate for the user based on the cardiovascular age, and calculating a fitness metric for the user based on the adjusted maximum heart rate.

19. A system, comprising:
a memory storing a latent space for an autoencoder that encodes a number of features of a photoplethysmography pulse signal based on a characteristic pulse shape of photoplethysmography pulse samples of a population, wherein the one or more features include an age associated with each of the photoplethysmography pulse samples of the population;
a wearable physiological monitor configured to acquire a sample of heart rate data from a user during a window including at least one characteristic pulse shape; and
one or more processors configured to perform the steps of:
receiving the sample,
encoding the sample with the autoencoder into the latent space,
identifying a cardiovascular age associated with the sample based on a location within the latent space, and
transmitting the cardiovascular age for presentation to the user.

20. The system of claim 19, wherein the one or more processors includes at least one of
a processor executing on a server remotely coupled to, and receiving data from, the wearable physiological monitor,
a processor executing on the wearable physiological monitor, and
a processor executing on a user computer locally coupled to the wearable physiological monitor.

21. The system of claim 19, wherein the latent space and the autoencoder are stored on the wearable physiological monitor.
